(19) United States
(12) Patent Application Publication
(10) Pub. No.: US 2024/0228990 A9
(43) Pub. Date: Jul. 11, 2024

(54) ENZYMES, MICRO-ORGANISMS AND USES THEREOF, AND A METHOD OF DEGRADING HYDROCARBON CHAINS

(71) Applicant: TEKNOLOGIAN TUTKIMUSKESKUS VTT OY, Espoo (FI)

(72) Inventors: Kari KOIVURANTA, Espoo (FI); Martina BLOMSTER ANDBERG, Espoo (FI); Heli NYGREN, Espoo (FI); Sandra CASTILLO, Espoo (FI)

(21) Appl. No.: 18/277,149

(22) PCT Filed: Feb. 15, 2022
(86) PCT No.: PCT/FI2022/050090

§ 371 Date: Aug. 14, 2023

(30) Foreign Application Priority Data
Feb. 16, 2021 (FI) 20215157

(51) Int. Cl. C12N 9/16; B09B 3/60; C08J 11/12; C12N 1/16; C12N 1/20; C12N 15/63

(52) U.S. Cl. CPC C12N 9/16; B09B 3/60; C08J 11/12; C12N 1/16; C12N 1/20; C12N 15/63; C12Y 301/00; C08J 2323/06; C12N 2800/101; C12N 2800/102

(57) ABSTRACT

The present invention relates to the fields of life sciences, micro-organisms and degradation of hydrocarbon chains such as plastics or synthetic polymers. Specifi-cally, the invention relates to an isolated specific enzyme, or a fragment thereof, wherein said enzyme or fragment is capable of degrading a hydrocarbon chain, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a hydrocarbon chain; to a method of degrading a hydrocarbon chain with the specific enzyme or a fragment thereof; and to a method of producing the enzyme or fragment thereof of the present invention.

### FIELD OF THE INVENTION

The present invention relates to the fields of life sciences, micro-organisms, and degradation of hydrocarbon chains such as plastics or synthetic polymers. Specifi-cally, the invention relates to an isolated specific enzyme, or a fragment thereof, wherein said enzyme or fragment is capable of degrading a hydrocarbon chain, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a hydrocarbon chain; to a method of degrading a hydrocarbon chain with the specific enzyme or a fragment thereof; and to a method of producing the enzyme or fragment thereof of the present invention.

### BACKGROUND OF THE INVENTION

With the existing plastic recycling systems (mechanical and chemical) not all plastic waste can be recycled. This is partly due to the quality of plastic wastes (mixed plastic, dirty plastics). Additionally, the existing recycling methods need much energy. Biotechnical recycling could be utilized for improving the range of recycling methods and for enabling cost effective and more efficient recycling of plastics.

Removal of highly stable and durable hydrocarbon chains including but not limited to plastics from the environment by using microbes or microbial enzymes is of high interest. In general, biotechnical plastic degradation is not common yet. Only few specific enzymes capable of degrading hydrocarbon chains or plastics have been discovered and said enzymes are not very effective. For example, Santo M. et al. (2013, International Biodeterioration & Biodegradation 84, 204-210) describe degradation of polyethylene (PE) with an extracellular fraction comprising different enzymes obtained from a Rhodococcus ruber cell culture. However, for PE or other hydrocarbon chains, recycling systems utilizing specific enzymes including but not limited to isolated and/or purified enzymes are under development. Indeed, it is very difficult to degrade hydrocarbon chains with enzymes.

Micro-organisms and enzymes are needed for rapid degradation and recycling of hydrocarbon chains. There remains a significant unmet need for specific micro-organisms and enzymes for effective degradation of hydrocarbon chains or plastics.

### BRIEF DESCRIPTION OF THE INVENTION

By biotechnical degradation and tools of the present invention it is possible to degrade and therefore recycle hydrocarbon chains such as plastics or synthetic polymers. Furthermore, the tools of the present invention can be used e.g. for upcycling hydrocarbon chains i.e. for modifying a non-biodegradable plastic (e.g. PE) to a biodegradable plastic (such as polyhydroxyalkanoate (PHA)) or fatty acid derived products (such as PHA and/or diacids) by micro-organisms and enzymes.

The objects of the invention, namely methods and tools for degrading hydrocarbon chains are achieved by utilizing a specific enzyme or enzymes, or a specific micro-organism or micro-organisms (e.g. a bacterium/bacteria and/or fungus/fungi) comprising said enzyme(s).

The methods and tools of present invention provide surprising degradation effects on hydrocarbon chains such as specific plastics or synthetic polymers, or a combination of specific plastics or synthetic polymers. The hydrocarbon chains to be degraded with the effective enzymes or micro-organisms of the present invention include but are not limited to high molecular weight hydrocarbon chains such as those comprised in long alkanes, alkenes, alcohols, aldehydes, ketones, polystyrene, polypropylene, and polyethylene, or on any combination thereof. Also, the present invention overcomes the problems of the prior art including but not limited to a slow biotechnical degradation speed. Actually, the present invention provides tools which enable biotechnical degradation of hydrocarbon chains, wherein the biotechnical degradation of said hydrocarbon chains has not been possible before.

The methods and tools of the present invention provide surprising degradation effects on hydrocarbon chains. Also, the present invention can overcome the problems of the prior art including but not limited to a slow biotechnical degradation speed.

Also, the inventors of the present disclosure surprisingly found out that unique or specific degradation products can be obtained with the present invention.

Novel biotechnical plastic recycling systems can be generated based on the enzyme or method of the present invention. The specific enzyme can be utilized in the degradation method e.g. at a temperature below 100° C. indicating low energy need.

Specifically, the present invention relates to a method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises one or more amino acids selected from the group comprising D23, H48, H50, D52, H53, H108 and D143 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17.

Also, the present invention relates to a method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises amino acids D23, H48, H50, D52, H53, H108 and D143 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17.

Also, the present invention relates to an isolated enzyme or a fragment thereof comprising one or more amino acids selected from the group comprising D23, H48, H50, D52, H53, H108 and D143 corresponding to the amino acid positions presented in SEQ ID NO: 2, and/or having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, wherein said enzyme or fragment is capable of degrading a hydrocarbon chain.

Also, the present invention relates to an isolated enzyme or a fragment thereof comprising amino acids D23, H48, H50, D52, H53, H108 and D143 corresponding to the amino acid positions presented in SEQ ID NO: 2, and/or having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, wherein said enzyme or fragment is capable of degrading a hydrocarbon chain.

Furthermore, the present invention relates to a micro-organism or a host cell comprising an enzyme or a fragment thereof comprising one or more amino acids selected from the group comprising D23, H48, H50, D52, H53, H108 and D143 corresponding to the amino acid positions presented in SEQ ID NO: 2, and/or having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, wherein said enzyme or fragment is capable of degrading a hydrocarbon chain.

Furthermore, the present invention relates to a micro-organism or a host cell comprising an enzyme or a fragment thereof comprising amino acids D23, H48, H50, D52, H53, H108 and D143 corresponding to the amino acid positions presented in SEQ ID NO: 2, and/or having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, wherein said enzyme or fragment is capable of degrading a hydrocarbon chain.

Still, the present invention relates to a polynucleotide encoding the enzyme or fragment thereof of the present invention.

Still, the present invention relates to an expression vector or plasmid comprising the polynucleotide of the present invention.

And still, the present invention relates to use of the enzyme, fragment, micro-organism, host cell, polynucleotide, expression vector or plasmid of the present invention or any combination thereof for degrading a hydrocarbon chain.

Still furthermore, the present invention relates to a method of producing the enzyme or fragment thereof of the present invention, wherein a recombinant micro-organism or host cell comprising the polynucleotide encoding the enzyme or fragment thereof of the present invention is allowed to express said enzyme or fragment thereof.

Other objects, details and advantages of the present invention will become apparent from the following drawings, detailed description, and examples.

The objects of the invention are achieved by methods, enzymes, fragments, micro-organisms, host cells, polynucleotides, vectors, plasmids and uses characterized by what is stated in the independent claims. The preferred embodiments of the invention are disclosed in the dependent claims.

### BRIEF DESCRIPTION OF THE DRAWINGS

FIG. 1 shows results from the GC-MS run. With Bacillus licheniformis enzyme samples and Zn and Cu several peaks appeared which were missing from control samples (in controls an empty plasmid) with polyethylene powder.

FIG. 2 shows results from the GC-MS run. With Bacillus licheniformis enzyme samples and Zn several peaks appeared which were missing from control samples (in control an empty plasmid) with LDPE film.

FIG. 3 shows results from the GC-MS run. With Bacillus flexus enzyme sample without added metal several peaks appeared which were not seen in control sample (in control an empty plasmid) with polyethylene powder.

FIG. 4 shows results from the GC-MS run. With Bacillus subtilis and Bacillus cereus enzyme samples and Zn several peaks appeared which were missing from control samples (in control an empty plasmid) with polyethylene powder.

FIG. 5 shows results of the heat GPC with Bacillus licheniformis metal-dependent hydrolase. Shown values are based on polystyrene standard.

FIG. 6 shows an alignment of several consensus amino acids of micro-organisms based on Bacillus licheniformis metal-dependent hydrolase amino acid positions Asp23, Pro24, His48, His50, Asp52, His53, Asp56, Ala106, His108, Gly142, Asp143, Thr144, Met172 and His193.

FIG. 7 shows a pairwise alignment of Ralstonia sp. (SEQ ID NO: 60) and Bacillus licheniformis (SEQ ID NO: 2) metal dependent hydrolases. Detected consensus amino acids have been marked with bold.

FIG. 8 shows results from the GC-MS run. With Brevibacillus borstelensis enzyme samples and Ni several peaks appeared which were missing from control samples (in control an empty plasmid) with polyethylene powder.

FIG. 9 shows results from the GC-MS run. With Micrococcus lylae enzyme samples and Zn several peaks appeared which were missing from control samples (in control an empty plasmid) with polyethylene powder.

FIG. 10 shows a plasmid map of pPB083.

FIG. 11 shows results from the GC-MS run. With cell extract sample of Yarrowia lipolytica expressing Bacillus licheniformis metal dependent hydrolase enzyme and Zn several peaks appeared which were missing from control samples (in control wild type Yarrowia lipolytica) with polyethylene powder.

FIG. 12 shows results from the GC-MS run. With supernatant sample of Yarrowia lipolytica expressing Bacillus licheniformis metal dependent hydrolase enzyme and Zn several peaks appeared which were missing from control samples (in control wild type Yarrowia lipolytica) with polyethylene powder.

FIG. 13 shows two-dimensional structure (alfa helixes and beta sheets) of Bacillus licheniformis metal dependent hydrolase (SEQ ID NO: 2) and localisation of consensus amino acids. Alfa helixes are underlined and numbered with Arabic numbers. Beta sheets are in Italics and numbered with Roman numbers. Consensus amino acids are in bold.

### SEQUENCE LISTING

SEQ ID NO: 1: Bacillus licheniformis metal-dependent hydrolase nucleotide sequence;
SEQ ID NO: 2: Bacillus licheniformis metal-dependent hydrolase amino acid sequence;
SEQ ID NO: 3: Bacillus cereus metal-dependent hydrolase nucleotide sequence;
SEQ ID NO: 4: Bacillus cereus metal-dependent hydrolase amino acid sequence;
SEQ ID NO: 5: Bacillus flexus metal-dependent hydrolase nucleotide sequence;
SEQ ID NO: 6: Bacillus flexus metal-dependent hydrolase amino acid sequence;
SEQ ID NO: 7: Bacillus subtilis metal-dependent hydrolase nucleotide sequence;
SEQ ID NO: 8: Bacillus subtilis metal-dependent hydrolase amino acid sequence;
SEQ ID NO: 9: Bacillus cohnii metal-dependent hydrolase nucleotide sequence;
SEQ ID NO: 10: Bacillus cohnii metal-dependent hydrolase amino acid sequence;
SEQ ID NO: 11: Bacillus circulans metal-dependent hydrolase nucleotide sequence;
SEQ ID NO: 12: Bacillus circulans metal-dependent hydrolase amino acid sequence;
SEQ ID NO: 13: Bacillus licheniformis metal-dependent hydrolase nucleotide sequence codon optimised to Yarrowia lipolytica;
SEQ ID NO: 14: Brevibacillus borstelensis metal-dependent hydrolase nucleotide sequence optimised to Escherichia coli;
SEQ ID NO: 15: Brevibacillus borstelensis metal-dependent hydrolase amino acid sequence;
SEQ ID NO: 16: Micrococcus lylae metal-dependent hydrolase nucleotide sequence optimised to Escherichia coli;
SEQ ID NO: 17: Micrococcus lylae metal-dependent hydrolase amino acid sequence;
SEQ ID NO: 18: oligonucleotide oPlastBug-106;
SEQ ID NO: 19: oligonucleotide oPlastBug-107;
SEQ ID NO: 20: oligonucleotide oPlastBug-140;
SEQ ID NO: 21: oligonucleotide oPlastBug-141;
SEQ ID NO: 22: oligonucleotide oPlastBug-220;
SEQ ID NO: 23: oligonucleotide oPlastBug-221;
SEQ ID NO: 24: oligonucleotide oPlastBug-110;
SEQ ID NO: 25: oligonucleotide oPlastBug-111;
SEQ ID NO: 26: Acinetobacter sp. metal-dependent hydrolase amino acid sequence;
SEQ ID NO: 27: Aneurinibacillus aneurinolyticus metal-dependent hydrolase amino acid sequence;
SEQ ID NO: 28: Bacillus agri metal-dependent hydrolase amino acid sequence;
SEQ ID NO: 29: Bacillus amyloliquefaciens metal-dependent hydrolase amino acid sequence;
SEQ ID NO: 30: Bacillus aryabhattai metal-dependent hydrolase amino acid sequence;
SEQ ID NO: 31: Bacillus mycoides metal-dependent hydrolase amino acid sequence;
SEQ ID NO: 32: Bacillus pumilus metal-dependent hydrolase amino acid sequence;
SEQ ID NO: 33: Bacillus vallismortis metal-dependent hydrolase amino acid sequence;
SEQ ID NO: 34: Bacillus vietnamensis metal-dependent hydrolase amino acid sequence;
SEQ ID NO: 35: Brevibacillus brevis metal-dependent hydrolase amino acid sequence;
SEQ ID NO: 36: Brevibacillus thuringiensis metal-dependent hydrolase amino acid sequence;
SEQ ID NO: 37: Chitinophaga sp. metal-dependent hydrolase amino acid sequence;
SEQ ID NO: 38: Cupriavidus necator metal-dependent hydrolase amino acid sequence;
SEQ ID NO: 39: Flavobacterium anhuiense metal-dependent hydrolase amino acid sequence;
SEQ ID NO: 40: Flavobacterium crocinum metal-dependent hydrolase amino acid sequence;
SEQ ID NO: 41: Flavobacterium sp. metal-dependent hydrolase amino acid sequence;
SEQ ID NO: 42: Flavobacterium succinicans metal-dependent hydrolase amino acid sequence;
SEQ ID NO: 43: Flavobacterium ummariense metal-dependent hydrolase amino acid sequence;
SEQ ID NO: 44: Flavobacterium xanthum metal-dependent hydrolase amino acid sequence;
SEQ ID NO: 45: Hyphomicrobium sp. metal-dependent hydrolase amino acid sequence;
SEQ ID NO: 46: Lysinibacillus fusiformis metal-dependent hydrolase amino acid sequence;
SEQ ID NO: 47: Lysinibacillus mangiferihumi metal-dependent hydrolase amino acid sequence;
SEQ ID NO: 48: Lysinibacillus sphaericus metal-dependent hydrolase amino acid sequence;
SEQ ID NO: 49 Lysinibacillus xylanilyticus metal-dependent hydrolase amino acid sequence;
SEQ ID NO: 50: Macrococcus caseolyticus metal-dependent hydrolase amino acid sequence;
SEQ ID NO: 51: Methylobacterium sp. metal-dependent hydrolase amino acid sequence;
SEQ ID NO: 52: Microbacterium sp. metal-dependent hydrolase amino acid sequence;
SEQ ID NO: 53: Nesiotobacter exalbescens metal-dependent hydrolase amino acid sequence;
SEQ ID NO: 54: Ochrobactrum_intermedium metal-dependent hydrolase amino acid sequence;
SEQ ID NO: 55: Ochrobactrum oryzae metal-dependent hydrolase amino acid sequence;
SEQ ID NO: 56: Paenibacillus macerans metal-dependent hydrolase amino acid sequence;
SEQ ID NO: 57: Paenibacillus sp. metal-dependent hydrolase amino acid sequence;
SEQ ID NO: 58: Paracoccus yeei metal-dependent hydrolase amino acid sequence;
SEQ ID NO: 59: Pseudomonas sp. metal-dependent hydrolase amino acid sequence;
SEQ ID NO: 60: Ralstonia sp. metal-dependent hydrolase amino acid sequence;
SEQ ID NO: 61: Rhodococcus sp. metal-dependent hydrolase amino acid sequence;
SEQ ID NO: 62: Staphylococcus cohnii metal-dependent hydrolase amino acid sequence;
SEQ ID NO: 63: Staphylococcus epidermidis metal-dependent hydrolase amino acid sequence;
SEQ ID NO: 64: Staphylococcus xylosus metal-dependent hydrolase amino acid sequence;
SEQ ID NO: 65: Streptomyces sp. metal-dependent hydrolase amino acid sequence;
SEQ ID NO: 66: Xanthobacter autotrophicus metal-dependent hydrolase amino acid sequence.

### DETAILED DESCRIPTION OF THE INVENTION

The present invention concerns a method of degrading a hydrocarbon chain, wherein a specific enzyme or micro-organism of the present invention is used for degrading said hydrocarbon chain. In one embodiment of the present invention a hydrocarbon chain or a material comprising one or more hydrocarbon chains (such as plastics or polymers of fossil origin, bio-based polymers or plastic material, polymer composites, copolymers, packaging material, textile, plastics or synthetic polymers (e.g. oil-based and/or biobased) containing waste material) is allowed to contact with an enzyme or micro-organism capable of degrading the hydrocarbon chain(s). In one embodiment of the invention the material comprising one or more hydrocarbon chains is a recycled material or from a recycled material.

As used herein “a hydrocarbon chain” refers to an organic compound, which comprises or consists of a chain of hydrogens and carbons (e.g. at least 4 C). In one embodiment the chain of hydrogens and carbons is linear, acyclic, cyclic, branched, aliphatic and/or aromatic. Therefore, “a hydrocarbon chain” refers e.g. to a hydrocarbon or a chain comprising a hydrocarbon chain like structure e.g. in the other end or one end of the chain. For example, long alkanes, alkenes, fatty acids, alcohols, aldehydes and ketones (e.g. comprising at least 20 C hydrocarbon chain like structure in the other end of the chain) and other compounds comprising a long hydrocarbon chain (e.g. at least 20 C hydrocarbon) like structure are within the scope of “hydrocarbon chains”. Compounds comprising at least one long hydrocarbon chain (e.g. at least 20 C hydrocarbon) like structure can have been obtained e.g. by a polymerization reaction.

Hydrocarbons can be classified to saturated hydrocarbons, unsaturated hydrocarbons and aromatic hydrocarbons. Saturated hydrocarbons comprise single bonds and are saturated with hydrogen. The formula for acyclic saturated hydrocarbons (i.e. alkanes) is CₙH₂ₙ₊₂. The most general form of saturated hydrocarbons is CₙH₂ₙ₊₂₍₁₋ᵣ₎, wherein r is the number of rings. Unsaturated hydrocarbons have one or more double or triple bonds between carbon atoms. Unsaturated hydrocarbons with double bonds are called alkenes and unsaturated hydrocarbons comprising triple bonds are called alkynes. Those with one double bond have the formula CₙH₂ₙ (assuming non-cyclic structures). Those with one triple bond have the formula CₙH₂ₙ₋₂. Aromatic hydrocarbons (arenes) have at least one aromatic ring.

In one embodiment a hydrocarbon chain (e.g. a linear hydrocarbon chain) is selected from the group comprising or consisting of polymers (e.g. plastics such as polyethylene, polypropylene, polystyrene, or multilayer materials or mixtures of materials comprising synthetic polymers or plastics and furthermore one or more materials such as paper and/or cardboard); gases (e.g. 1,7-octadiene); and liquids (e.g. dodecane). In one embodiment a hydrocarbon chain (e.g. a chain or compound comprising a hydrocarbon like structure) is selected from the group comprising or consisting of a long ketone, long alkane, long alkene, long alkyne, long cycloalkane, long alkadiene, long fatty acid, long alcohol and long carbon chain aldehyde.

In one embodiment of the invention the hydrocarbon chain is a hydrocarbon chain of a synthetic polymer, alkane, alkene, alkyne, cycloalkane, alkadiene, ketone, fatty acid, alcohol, aldehyde, polyolefin, polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low density polyethylene (VLDPE), or any combination thereof.

In one embodiment a long hydrocarbon chain or a long hydrocarbon chain like structure has a chain length of at least C20, C22, C24, C26, C28, C30, C32, C34, C36, C38, C40, C45, C50, C60, C70, C80, C90 or C100. In one embodiment, the length of the hydrocarbon chain degraded or degradable by the present invention is at least C20, C22, C24, C26, C28, C30, C32, C34, C36, C38, C40, C45, C50, C60, C70, C80, C90, C100, C150, C200, C250, C300, C350, C400, C450 or C500.

As used herein, “a plastic” refers to a material comprising or consisting of synthetic and/or semi-synthetic organic compounds and having the capability of being molded or shaped. As used herein “a synthetic polymer” refers to a human-made polymer. Synthetic polymers can be classified into four main categories: thermoplastics, thermosets, elastomers, and synthetic fibers. Thermoplastics are a type of synthetic polymers that become moldable and malleable past a certain temperature, and they solidify upon cooling. Thermosets become hard and cannot change shape once they have set. Elastomers are flexible polymers. Synthetic fibers are fibers made by humans through a chemical synthesis.

As used herein polyolefin refers to a type of polymer produced from a simple olefin (also called an alkene with the general formula CₙH₂ₙ) as a monomer. For example, polyethylene and polypropylene are common polyolefins. Depending on a polymerization method utilized for producing a polyolefin hydrocarbon chain, the polyolefin hydrocarbon chain sometimes comprises a specific group or groups such as a ketone group e.g. at the end of the chain.

Polyethylene (PE) consists of long chain polymers of ethylene and it is produced as either high-density (HD-PE) or low-density polyethylene (LD-PE). PE is chemically synthesized by polymerization of ethane and is highly variable, since side chains can be obtained depending on the manufacturing process. In one embodiment LDPE is defined by a density range of about 910-930 kg/m³, and/or the density range of HDPE is about 930 to 970 kg/m³.

Cross-linked polyethylene (PEX or XLPE) is a form of polyethylene with cross-linked bonds in the polymer structure, changing the thermoplastic to a thermoset. Indeed, crosslinking enhances the temperature properties of the base polymer and furthermore e.g. tensile strength, scratch resistance, and resistance to brittle fracture.

Ultra-high molecular weight polyethylene (UHMWPE) is a type of polyolefin and a subset of the thermoplastic polyethylene. It is made up of extremely long chains of polyethylene, which all align in the same direction. The extremely long chain can usually have a molecular mass between 3.5 and 7.5 million amu.

High-density polyethylene (HDPE) is a thermoplastic polymer produced from the monomer ethylene. The density of HDPE can range from 930 to 970 kg/m³.

Medium-density polyethylene (MDPE) is a type of polyethylene and can be defined e.g. by a density range of 0.926-0.940 g/cm³.

Low density polyethylene (LDPE) has more branching than HDPE (i.e. has a high degree of short- and long-chain branching), and therefore it's intermolecular forces are weaker, its tensile strength is lower, and its resilience is higher. Also, because its molecules are less tightly packed and less crystalline due to the side branches, its density is lower. LDPE can be defined by a density range of 0.910-0.930 g/cm³.

Linear low-density polyethylene (LLDPE) is a substantially linear polyethylene with significant numbers of short branches. LLDPE differs structurally from conventional LDPE because of the absence of long chain branching.

Very low density polyethylene (VLDPE) is a type of LLDPE with higher levels of short-chain branches than standard LLDPE. VLDPE can be defined by a density range of 0.880-0.910 g/cm³.

In one embodiment of the invention the enzyme capable of degrading a hydrocarbon chain or a hydrocarbon chain containing material is from a bacterium (gram-positive or gram-negative) or fungus, and/or the micro-organism capable of degrading a hydrocarbon chain or a hydrocarbon chain containing material is a bacterium (gram-positive or gram-negative) or fungus.

As used herein, “degradation” of a hydrocarbon chain, plastic, synthetic or non-synthetic polymer refers to either partial or complete degradation of a hydrocarbon chain, plastic, synthetic or non-synthetic polymer to a shorter hydrocarbon chain (such as a hydrocarbon chain comprising one or more organic compounds, a long ketone, a long alcohol, a long fatty acid), oligomers and/or monomers. Said degradation can also include lowering of the molecular weight of a hydrocarbon chain or polymer, lowering of the average molecular weight, lowering of the molar mass in the peak of maximum and/or increase in polydispersity of a hydrocarbon chain or polymer. Indeed, any loss in the chain length of a hydrocarbon chain or polymer can e.g. lower tensile strength. “Enzymatic or microbial degradation” refers to a degradation caused by an enzyme or micro-organism, respectively. According to some hypothesis, in the microbial degradation the larger polymers are initially degraded by secreted exoenzymes or by outer membrane bound enzymes into smaller subunits (different length oligomers) that can be incorporated into the cells of micro-organisms and further degraded through the classical degradation pathways to yield energy and/or suit as building blocks for catabolism or metabolism. Many plastics or other materials are mixtures comprising synthetic or semi-synthetic polymers and furthermore solubilizers and optionally other chemical agents for altering the mechanical and physical properties of said plastics or materials. The solubilizers and other chemical compounds may also be targets of enzymatic or microbial biodegradation.

In one embodiment of the invention the enzyme (or a fragment thereof), micro-organism or host cell comprises alkane, alkene, ketone, fatty acid, alcohol, aldehyde, polyolefin, PE, PEX, UHMWPE, HDPE, MDPE, LLDPE, LDPE, and/or VLDPE degrading activity, or any combination thereof. In one embodiment the enzymes, fragments, micro-organisms or host cells of the present invention can be capable of utilizing short, medium-sized and/or long hydrocarbon chain substrates (such as those having a molecular weight of 100 Da-50 000 kDa, e.g. 5 000 Da-10 000 kDa).

Degradation of a hydrocarbon chain, synthetic polymer or plastic can result in at least one or more degradation products. In one embodiment of the invention, at least one or more degradation products selected from the group consisting of an alkane, alkene, alkyne, cycloalkane, alkadiene, ketone (e.g. ketone C2-C32), fatty acid, alcohol, aldehyde, epoxy, benzene, styrene, diacid, 2-decanone, 2-dodecanone, 2-tetradecanone, 2-hexadecanone, 2-heptadecanone and 2-dotriacontanone are obtained or obtainable by the degradation of the hydrocarbon chain. For example, PE can be degraded to an alkane, alkene, alkyne, cycloalkane, alkadiene, ketone (e.g. ketone C2-C32), fatty acid, alcohol, aldehyde, diacid, 2-decanone, 2-dodecanone, 2-tetradecanone, 2-hexadecanone, 2-heptadecanone and/or 2-dotriacontanone. Thus, in one embodiment, the method of degrading a hydrocarbon chain comprises obtaining, recovering, removing, recycling and/or re-utilizing at least one of the degradation products.

In one embodiment of the invention only the enzyme(s) or micro-organism(s) or a combination thereof is(are) needed for a biotechnical or enzymatic degradation of a hydrocarbon chain or a combination of different types of hydrocarbon chains. In other words, no other degradation methods such as UV light or mechanical disruption or chemical degradation are needed in said embodiment. In other embodiments, biotechnical, enzymatic or microbial degradation can be combined with one or more other degradation methods (e.g. non-enzymatic degradation methods) including but not limited to UV light, gamma irradiation, microwave treatment, mechanical disruption and/or chemical degradation. In one embodiment of the invention the method of degrading a hydrocarbon chain is a biotechnical method, or the method comprises degradation of the hydrocarbon chain by non-enzymatic methods or means. Non-enzymatic, non-microbial or non-biotechnical degradation methods or steps including pretreatments can be carried out sequentially (e.g. before or after) or simultaneously with the biotechnical, microbial or enzymatic degradation.

The present invention concerns an isolated enzyme or a fragment thereof comprising one or more amino acids selected from the group comprising or consisting of D23, P24, H48, H50, D52, D56, H53, A106, H108, G142, D143, T144, M172 and H193, corresponding to the amino acid positions presented in SEQ ID NO: 2, wherein said enzyme or fragment is capable of degrading a hydrocarbon chain. Also, the present invention concerns a micro-organism or a host cell comprising an enzyme or a fragment thereof comprising one or more amino acids selected from the group comprising or consisting of D23, P24, H48, H50, D52, D56, H53, A106, H108, G142, D143, T144, M172 and H193 corresponding to the amino acid positions presented in SEQ ID NO: 2, wherein said enzyme or fragment is capable of degrading a hydrocarbon chain.

More specifically, the present invention concerns an isolated enzyme or a fragment thereof comprising amino acids D23, H48, H50, D52, H53, H108, and D143 corresponding to the amino acid positions presented in SEQ ID NO: 2, wherein said enzyme or fragment is capable of degrading a hydrocarbon chain. Also, the present invention concerns a micro-organism or a host cell comprising an enzyme or a fragment thereof comprising amino acids D23, H48, H50, D52, H53, H108, and D143 corresponding to the amino acid positions presented in SEQ ID NO: 2, wherein said enzyme or fragment is capable of degrading a hydrocarbon chain.

The enzyme of the present invention refers to not only fungal or bacterial but also any other enzyme homologue from any micro-organism, organism or mammal. Also, all isozymes, isoforms and variants are included with the scope of said enzyme. In one embodiment of the method, enzyme, fragment, micro-organism or host cell of the present invention, the enzyme originates from or is an enzyme of a bacterium selected from the group comprising or consisting of Bacillus, Paenibacillus, Achromobacter, Acinetobacter, Alcanivorax, Aneurinibacillus, Arthrobacter, Brevibacillus, Chitinophaga, Citrobacter, Cupriavidus, Delftia, Enterobacter, Flavobacterium, Hyphomicrobium, Klebsiella, Kocuria, Leucobacter, Lysinibacillus, Macrococcus, Methylobacterium, Methylocella, Microbacterium, Micrococcus, Moraxella, Nesiotobacter, Nocardia, Ochrobactrum, Pantoea, Paracoccus, Pseudomonas, Rahnella, Ralstonia, Rhizobium, Rhodococcus, Serratia, Staphylococcus, Stenotrophomonas, Streptomyces, Vibrio, Virgibacillus and Xanthobacter; or the enzyme is an enzyme of a bacterium selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Chitinophaga sp., Citrobacter amalonaticus, Cupriavidus necator, Delftia sp., Delftia tsuruhatensis, Enterobacter sp., Flavobacterium sp., Flavobacterium petrolei, Flavobacterium pectinovorum, Flavobacterium aquicola, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus lylae, Moraxella sp., Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Pseudomonas aeruginosa, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptomyces albogriseolus, Streptomyces badius, Streptomyces griseus, Streptomyces sp., Streptomyces viridosporus, Vibrio alginolyticus, Vibrio parahaemolyticus, Virgibacillus halodenitrificans, Xanthobacter autotrophicus, and Xanthobacter tagetidis.

In one embodiment “an enzyme of a bacterium” refers to a situation, wherein the amino acid sequence of the enzyme has the same amino acid sequence as a wild type enzyme of a bacterium (e.g. any of the above listed bacteria) or the amino acid sequence of the enzyme has a high sequence identity (e.g. 50% or more, 60% or more, 70% or more, 80% or more, 85% or more, 90% or more, or 95% or more) to an amino acid sequence of a wild type bacterial enzyme (e.g. of any of the above listed bacteria). In other words, the amino acid sequence of the enzyme used in the present invention can be modified (e.g. genetically modified).

In one embodiment, the enzyme, fragment, micro-organism or host cell is a genetically modified enzyme, fragment, micro-organism or host cell. In a specific embodiment the enzyme, fragment, micro-organism or host cell has an increased ability to degrade a hydrocarbon chain compared to the corresponding unmodified enzyme, fragment, micro-organism or host cell, respectively. In one embodiment the enzyme, micro-organism or host cell comprises a genetic modification increasing an enzyme activity or the amount of a specific enzyme in a micro-organism or host cell. Genetic modifications (e.g. resulting in increased enzyme activity, increased expression of an enzyme, or increased or faster degradation of a hydrocarbon chain) include but are not limited to genetic insertions, deletions, disruptions or substitutions of one or more genes or a fragment(s) thereof or insertions, deletions, disruptions or substitutions of one or more nucleotides (e.g. insertion of a polynucleotide encoding an enzyme), or addition of plasmids. For example, one or several polynucleotides encoding an enzyme of interest can be integrated to the genome of a micro-organism or host cell. As used herein “disruption” refers to insertion of one or several nucleotides into a gene or polynucleotide sequence resulting in a lack of the corresponding polypeptide or enzyme or presence of non-functional polypeptide or enzyme with lowered activity. Methods for making any genetic modifications or modifying micro-organisms or host cells (e.g. by adaptive evolution strategy) are generally well known by a person skilled in the art and are described in various practical manuals describing laboratory molecular techniques.

In one embodiment the enzyme or a fragment thereof has one or more genetic modifications (e.g. a targeted mutation or a modification by an adaptive evolution) after one or more amino acids corresponding to the amino acids selected from the group comprising or consisting of D23, P24, H48, H50, D52, D56, H53, A106, H108, G142, D143, T144, M172 and H193 presented in SEQ ID NO: 2. As used herein “after one or more amino acids” refers to immediately after said amino acid(s) e.g. a modification at least in the next amino acid or later after said amino acid (e.g. 1-50 amino acids, 1-30 amino acids, 1-20 amino acids, 1-10 amino acids or 1-5 amino acids after the specific amino acid mentioned above in the list of this paragraph).

As used herein “increased degradation (activity/ability/capability) of a hydrocarbon chain” or “faster degradation (activity/ability/capability) of a hydrocarbon chain” of an enzyme or micro-organism refers to the presence of higher activity or more activity of an enzyme or micro-organism, when compared to another enzyme or micro-organism, e.g. a genetically unmodified (wild type) enzyme or micro-organism. “Increased or faster degradation” may result e.g. from the presence of a specific enzyme in a micro-organism or an up-regulated gene or polypeptide expression in a micro-organism or an increased secretion of an enzyme by a micro-organism. Also, “increased or faster degradation” may result e.g. from the presence of (enhancing) mutations of a specific enzyme having degradation capability.

As used herein “up-regulation of the gene or polypeptide expression” refers to excessive expression of a gene or polypeptide by producing more products (e.g. mRNA or polypeptide, respectively) than an unmodified micro-organism. For example, one or more copies of a gene or genes may be transformed to a cell (e.g. to be integrated to the genome of the cell) for upregulated gene expression. The term also encompasses embodiments, where a regulating region such as a promoter or promoter region has been modified or changed or a regulating region (e.g. a promoter) not naturally present in the micro-organism has been inserted to allow the over-expression of a gene. Also, epigenetic modifications such as reducing DNA methylation or histone modifications as well as classical mutagenesis are included in “genetic modifications”, which can result in an upregulated expression of a gene or polypeptide. As used herein “increased or up-regulated expression” refers to an increased expression of the gene or polypeptide of interest compared to a wild type micro-organism without the genetic modification. Expression or increased expression can be proved for example by western, northern or southern blotting or quantitative PCR or any other suitable method known to a person skilled in the art. As used herein “increased secretion of an enzyme by a micro-organism” refers to a secretion of an enzyme outside of a cell, which produces said enzyme. Increased secretion may be caused e.g. by an increased or upregulated expression of the gene or polypeptide of interest or by improved secretion pathway of the cell or molecules participating in the secretion of said enzyme.

In one embodiment the genetically modified enzyme, micro-organism, host cell or polynucleotide is a recombinant enzyme, micro-organism, host cell or polynucleotide. As used herein, “a recombinant enzyme, micro-organism, host cell or polynucleotide” refers to any enzyme, micro-organism, host cell or polynucleotide that has been genetically modified to contain different genetic material compared to the enzyme, micro-organism, host cell or polynucleotide before modification (e.g. comprise a deletion, substitution, disruption or insertion of one or more nucleic acids or amino acids e.g. including an entire gene(s) or parts thereof). The recombinant micro-organism or host cell may also contain other genetic modifications than those specifically mentioned or described in the present disclosure. Indeed, the micro-organism or host cell may be genetically modified to produce, not to produce, increase production or decrease production of e.g. other polynucleotides, polypeptides, enzymes or compounds than those specifically mentioned in the present disclosure. In certain embodiments, the genetically modified micro-organism or host cell includes a heterologous polynucleotide or enzyme. The micro-organism or host cell can be genetically modified by transforming it with a heterologous polynucleotide sequence that encodes a heterologous polypeptide. For example, a cell may be transformed with a heterologous polynucleotide encoding an enzyme of the present invention either without a signal sequence or with a signal sequence. Alternatively, for example heterologous promoters or other regulating sequences can be utilized in the micro-organisms, host cells or polynucleotides of the invention. As used herein “a heterologous polynucleotide or enzyme” refers to a polynucleotide or enzyme, which does not naturally occur in a cell or micro-organism. In one embodiment of the present invention, the enzyme or fragment thereof is encoded by a heterologous polynucleotide sequence and optionally expressed by a micro-organism or host cell.

Genetic modifications may be carried out using conventional molecular biological methods. Genetic modification (e.g. of an enzyme or micro-organism) can be accomplished in one or more steps via the design and construction of appropriate vectors and transformation of the micro-organism cell with those vectors. For example, electroporation, protoplast-PEG and/or chemical (such as calcium chloride or lithium acetate based) transformation methods can be used. Also, any commercial transformation methods are appropriate. Suitable transformation methods are well known to a person skilled in the art.

The term “vector” refers to a nucleic acid compound and/or composition that transduces, transforms, or infects a micro-organism or a host cell, thereby causing the cell to express polynucleotides and/or proteins other than those native to the cell, or in a manner not native to the cell. An “expression vector” contains a sequence of nucleic acids to be expressed by the modified micro-organism. Optionally, the expression vector also comprises materials to aid in achieving entry of the nucleic acids into the micro-organism, such as a virus, liposome, protein coating, or the like. The expression vectors contemplated for use in the present invention include those into which a nucleic acid sequence (i.e. polynucleotide) can be inserted, along with any preferred or required operational elements. Further, the expression vector must be one that can be transferred into a micro-organism or host cell and replicated therein. Vectors can be circularized or linearized and may contain restriction sites of various types for linearization or fragmentation. In specific embodiments expression vectors are plasmids, particularly those with restriction sites that have been well documented and that contain the operational elements preferred or required for transcription of the nucleic acid sequence. Such plasmids, as well as other expression vectors, are well known to those of ordinary skill in the art. Useful vectors may for example be conveniently obtained from commercially available micro-organism, yeast or bacterial vectors. Successful transformants can be selected using the attributes contributed by the marker or selection gene. Screening can be performed e.g. by PCR or Southern analysis to confirm that the desired genetic modifications (e.g. deletions, substitutions or insertions) have taken place, to confirm copy number or to identify the point of integration of nucleic acids (i.e. polynucleotides) or genes into the micro-organism cell's genome.

Indeed, the present invention also relates to a polynucleotide encoding the enzyme of the present invention or a fragment thereof, and an expression vector or plasmid comprising said polynucleotide of the present invention.

In a specific embodiment the enzyme of the present invention comprises or has a sequence having at least 20%, 25%, 30%, 35%, 40%, 45%, 50%, 55%, 60%, 65%, 70%, 71%, 72%, 73%, 74%, 75%, 76%, 77%, 78%, 79%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99% (e.g. 99.1%, 99.2%, 99.3%, 99.4%, 99.5%, 99.6%, 99.7%, 99.8% or 99.9%) or 100% sequence identity to SEQ ID NO: 2, 4, 6, 8, 10, 12, 15, or 17, or an enzymatically active fragment or variant thereof. Said enzyme can be genetically modified (i.e. differs from the wild type enzyme) or unmodified. In a specific embodiment an enzyme is an isolated enzyme.

In one embodiment of the invention the enzyme has at least 20, 25, 26, 27, 28, 29, 30, 35, 40, 45, 50, 55, 60, 65, 70, 71, 72, 73, 74, 75, 76, 77, 78, 79, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99 (e.g. 99.1, 99.2, 99.3, 99.4, 99.5, 99.6, 99.7, 99.8 or 99.9%), or 100% sequence identity to SEQ ID NO: 2 (SEQ ID NO: 2 is a Bacillus licheniformis metal-dependent hydrolase amino acid sequence).

In one embodiment, the enzyme or fragment comprises a signal sequence, e.g. a heterologous signal sequence or a signal sequence of an exogenous host cell producing said enzyme of a fragment thereof. The signal sequence can be located e.g. after or before the amino acid sequence of the enzyme e.g. for secreting said enzyme outside of the cell. The signal sequence can be any signal sequence i.e. a short polypeptide present at the N-terminus of synthesized polypeptides that are destined towards the secretory pathway, said polypeptides including but not limited to those polypeptides that are targeted inside specific organelles, secreted from the cell, or inserted into cellular membranes. A signal sequence for secreted extracellular proteins can be predicted e.g. by using prediction tools like SignalP-5.0 (https://services.healthtech.dtu.dk/service.php?SignalP-5.0). In one embodiment the enzyme or fragment thereof comprises a signal sequence, does not comprise a detectable signal sequence, is secreted out of the cell which produces it, and/or is not secreted out of the cell which produces it. In one embodiment the enzyme or fragment thereof does not comprise a detectable signal sequence and is secreted out of the cell which produces it.

A polynucleotide of the present invention encodes the enzyme of the present invention or a fragment thereof. In a specific embodiment the polynucleotide comprises a sequence having a sequence identity of at least 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to SEQ ID NO: 1, 3, 5, 7, 9, 11, 13, 14, or 16, or a variant thereof. Said polynucleotide can be genetically modified (i.e. differs from the wild type polynucleotide) or unmodified. In a specific embodiment the polynucleotide is an isolated polynucleotide.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e., identity=#of identical positions/total #of positions×100), taking into account the number of gaps, and the length of each gap, which need to be introduced for optimal alignment of the two sequences. The comparison of sequences and determination of identity percentage between two sequences can be accomplished using mathematical algorithms available in the art. This applies to both amino acid and nucleic acid sequences. As an example, sequence identity may be determined by using BLAST (Basic Local Alignment Search Tools) or FASTA (FAST-All). In the searches, setting parameters “gap penalties” and “matrix” are typically selected as default. In one embodiment the sequence identity is determined against the full length sequence of the present disclosure.

Nucleic acid and amino acid databases (e.g., GenBank) can be used for identifying a polypeptide having an enzymatic activity or a polynucleotide sequence encoding said polypeptide. Sequence alignment software such as BLASTP (polypeptide), BLASTN (nucleotide) or FASTA can be used to compare various sequences. Briefly, any amino acid sequence having some homology to a polypeptide having enzymatic activity, or any nucleic acid sequence having some homology to a sequence encoding a polypeptide having enzymatic activity can be used as a query to search e.g. GenBank. Percent identity of sequences can conveniently be computed using BLAST software with default parameters. Sequences having an identities score and a positive score of a given percentage, using the BLAST algorithm with default parameters, are considered to be that percent identical or homologous.

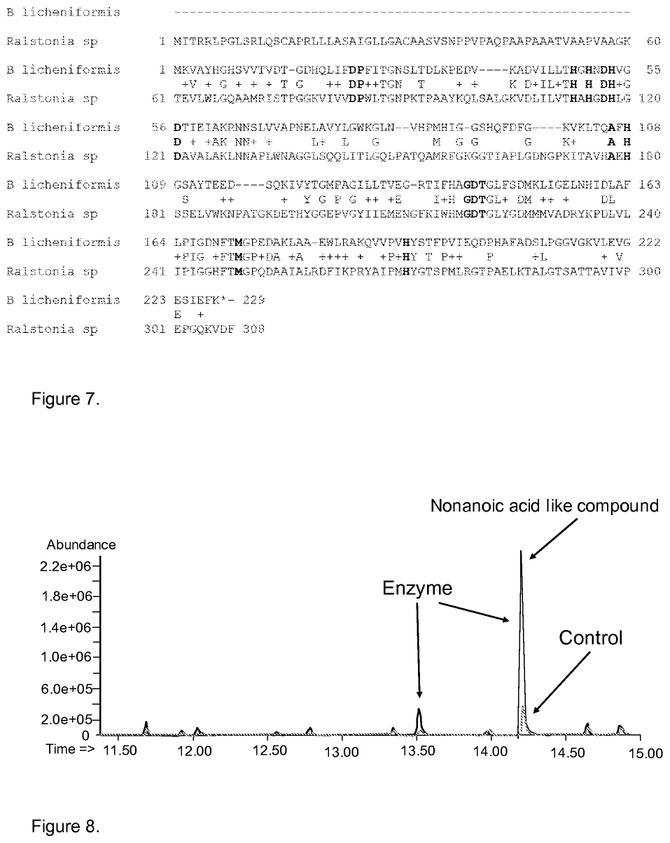

For example, an enzyme comprising a hydrocarbon chain degrading activity and e.g. comprising amino acids D23, H48, H50, D52, H53, H108 and D143 corresponding to the amino acid positions presented in SEQ ID NO: 2, can be found as described in example 7. First, sequences containing similar kind of motifs can be searched e.g. with HMMER. HMMER is used for searching sequence databases for sequence homologs, and for making sequence alignments. It implements methods using probabilistic models called profile hidden Markov models (profile HMMs) (Robert D. Finn, Jody Clements, Sean R. Eddy (2011) HMMER web server: interactive sequence similarity searching. Nucleic Acids Research, Volume 39, Issue suppl_2, 1 July 2011, Pages W29-W37, https://doi.org/10.1093/nar/gkr367). With the detected amino acid sequences or part of them or amino acid sequence(s) of previously known enzyme(s) sequence similarity searches against SEQ ID NO: 2 can be carried out e.g. by sequence alignment with ClustalW programme (httbs://www.genome.jp/tools-bin/clustalw) to detect corresponding consensus amino acids and their positions in amino acid sequence of interest. (See e.g. FIG. 7.)

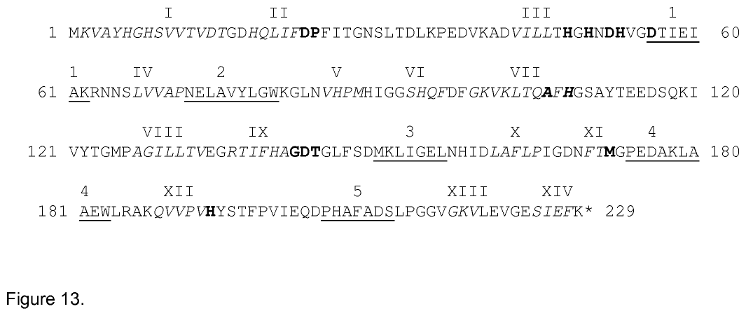

In one embodiment one or more of the amino acids D23, H48, H50, D52, H53, H108 and D143 (corresponding to the amino acid positions presented in SEQ ID NO: 2) are critical for the activity of the enzyme, e.g. degradation of a substrate. The enzyme can comprise one or more specific amino acids or amino acid motifs for example affecting a hydrocarbon chain degrading activity (e.g. enabling different substrates and/or binding of metal ions). In one embodiment of the method, enzyme, fragment, micro-organism or host cell of the present invention, the enzyme or fragment comprises one or several amino acids selected from the group comprising D23, H48, H50, D52, H53, H108 and D143, wherein the amino acids and positions correspond to the amino acids and positions presented in SEQ ID NO: 2. This means that the enzyme or fragment comprises one or several amino acids, which correspond to the amino acids D23, H48, H50, D52, H53, H108 and/or D143 as shown in SEQ ID NO: 2. In one embodiment of the method, enzyme, fragment, micro-organism or host cell of the present invention, the enzyme or fragment comprises the amino acids D23, H48, H50, D52, H53, H108 and D143, wherein the amino acids and positions correspond to the amino acids and positions presented in SEQ ID NO: 2. These amino acids seem to be critical to the metal binding, right protein structure and activity. These amino acids are located in the loop area (D23, H48, H50, D52, H53, D143) and in the beta sheet 7 (H108) (see FIG. 13).

In one embodiment the enzyme or fragment comprises one, several or all amino acids Asp23, Pro24, His48, His50, Asp52, His53, Asp56, Ala106, His108, Gly142, Asp143, Thr144, Met172 and His193, wherein the amino acids and positions correspond to the amino acids and positions presented in SEQ ID NO: 2. In one embodiment one or more of the consensus amino acids affect the degrading activity (e.g. by increasing the degrading activity) of hydrocarbon chains (e.g. Pro24), increase possible interaction with substrates (e.g. Asp23 and/or Asp143), or affect binding of a metal ion (e.g. His108). In one embodiment of the method, enzyme, fragment, micro-organism or host cell of the present invention, the enzyme or fragment comprises the amino acids D23, H48, H50, D52, H53, H108 and D143, and one, several or all amino acids Pro24, Asp56, Ala106, Gly142, Thr144, Met172 and His193, wherein the amino acids and positions correspond to the amino acids and positions presented in SEQ ID NO: 2. In one embodiment of the method, enzyme, fragment, micro-organism or host cell of the present invention, the enzyme or fragment comprises the amino acids D23, P24, H48, H50, D52, H53, D56, A106, H108, G142, D143, T144, M172 and H193, wherein the amino acids and positions correspond to the amino acids and positions presented in SEQ ID NO: 2. The amino acids Pro24, Gly142, Thr144, Met172 and His193 are located in the loop area, Asp56 in alpha helix 1 and Ala106 in the beta sheet 7 (see FIG. 13).

In one embodiment of the method, enzyme, fragment, micro-organism or host cell of the present invention the enzyme is selected from the group comprising or consisting of beta-lactamase, a hydrolase, metal-dependent hydrolase, and DNA polymerase, or any combination thereof; and/or the enzyme comprises betalactamase, hydrolase, metal-dependent hydrolase or DNA polymerase activity, or any combination thereof. As used herein “a beta-lactamase” refers to an enzyme that can provide antibiotic resistance by breaking the antibiotic's structure. As used herein “a hydrolase” refers to an enzyme which is capable of catalyzing the hydrolysis of a chemical bond optionally resulting in a degradation of a larger molecule into smaller molecules. Examples of hydrolases include but are not limited to esterases, lipases, phosphatases, glycosidases and peptidases. “A metal-dependent hydrolase” refers to an enzyme, which uses one or more metal ion co-factors in combination with amino acid side chains to catalyze hydrolysis of a wide variety of biologically important substrates, including but not limited to carbohydrates, peptides, nucleotides, phosphodiesters and xenobiotics. As used herein “a DNA polymerase” refers to an enzyme which catalyzes the synthesis of DNA molecules from nucleoside triphosphates. DNA polymerases are essential for DNA replication because they create two identical DNA duplexes from a single original DNA duplex. The enzyme(s) involved in the degradation of hydrocarbon chains can be selected e.g. from one or several of the following: a hydrolase (EC 3), a metal dependent hydrolase (e.g. EC 3.1, EC 3.4 or EC 3.5), a carboxylic ester hydrolase (e.g. EC 3.1.1, e.g. arylesterase EC 3.1.1.2 or cutinase EC 3.1.1.74), an amidohydrolase (e.g. EC 3.5.1 or EC 3.5.2, e.g. beta-lactamase EC 3.5.2.6), a hydrolase acting on carbon-carbon bonds (e.g. EC 3.7) and a DNA polymerase (e.g. EC 2.7.7.7).

In one embodiment the enzyme is capable of binding a divalent metal ion. In one embodiment the divalent metal ion is Zn2+, Cu2+, Ca2+, Ni2+, Mn2+, Co2+, Fe2+, Mg2+, Cd2+, or any combination thereof. For example, the enzyme can bind at least Cu2+, Co2+and Fe2+; and/or Zn2+and Cu2+. In one embodiment of the invention a divalent metal ion is part of the structure of the enzyme. In that case the enzyme cannot bind a divalent metal ion added to the culture.

In one embodiment the enzyme and/or micro-organism have been genetically modified and optionally have an increased ability to degrade a hydrocarbon chain compared to the corresponding unmodified enzyme and/or micro-organism, respectively.

The presence, absence or amount of specific enzyme activities can be detected by any suitable method known in the art. Specific examples of studying enzyme activities of interest are well known to a person skilled in the art. Non-limiting examples of suitable detection methods include commercial kits on market, enzymatic assays, immunological detection methods (e.g., antibodies specific for said proteins), PCR based assays (e.g., qPCR, RT-PCR), and any combination thereof.

In one embodiment, the enzymes of the present invention have high turnover rates when degrading one or more hydrocarbon chains, e.g. when compared to prior art enzymes. In specific embodiments the activity of an enzyme to degrade a hydrocarbon chain is determined by an enzyme assay wherein said enzyme is allowed to contact with hydrocarbon chains (e.g. as described in any of examples 4, 6, 9 and 10). In some embodiments the activity of an enzyme to degrade hydrocarbon chains can be determined e.g. by detecting or measuring the degradation products of hydrocarbon chains (e.g. as shown in example 5) or by analyzing the remaining starting material containing hydrocarbon chains after contacting the starting material with the enzymes (e.g. as shown in example 6).

Degradation of hydrocarbon chains can be measured by any suitable method known in the field. In one embodiment hydrocarbon chains or a material comprising hydrocarbon chains are weighed before and/or after said hydrocarbon chains or material have been contacted with an enzyme, micro-organism or host cell (or any combination thereof). The presence, absence or level of degradation products of a hydrocarbon chain, e.g. degraded by an enzyme, micro-organism or host cell, can be detected or measured by any suitable method known in the art. Non-limiting examples of suitable detection and/or measuring methods include liquid chromatography, gas chromatography, mass spectrometry or any combination thereof (e.g. ESI-MS/MS, MaldiTof, RP-HPLC, GC-MS or LC-TOF-MS) of samples, optionally after cultivating a micro-organism or host cell e.g. 1-11 hours, 11-100 hours, or 100 hours-12 months (e.g. one, two, three, four, five, six, seven, eight, nine, ten or 11 months) or even longer in the presence of hydrocarbon chains (such as plastics or synthetic polymers) or after allowing a micro-organism, polypeptide or enzyme to contact with hydrocarbon chains. Other examples of suitable detection and/or measuring methods (including methods of fractionating, isolating or purifying degradation products) include but are not limited to filtration, solvent extraction, centrifugation, affinity chromatography, ion exchange chromatography, electrophoresis, hydrophobic interaction chromatography, gel filtration chromatography, reverse phase chromatography, chromatofocusing, differential solubilization, preparative disc-gel electrophoresis, isoelectric focusing, HPLC, gel permeation chromatography (GPC), fourier-transform infrared spectroscopy (FTIR), NMR and/or reversed-phase HPLC.

For degradation, hydrocarbon chains or a material comprising hydrocarbon chains can be contacted with an enzyme, micro-organism or host cell (or any combination thereof) at a ratio, concentration and/or temperature for a time sufficient for the degradation of interest. Suitable time for allowing the enzyme, micro-organism, or host cell to degrade a hydrocarbon chain or hydrocarbon chains can be selected e.g. from the group consisting of 1, 2, 3, 4, 5, 6, 7, 8, 9, 10, 11, 12, 13, 14, 15, 16, 17, 18, 19, 20, 21, 22, 23, and 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 days, 1, 2, 3, 4, 5, 6, 7, 8, 9, 10, 11, and 12 weeks, and 1, 2, 3, 4, 5, 6, 7, 8, 9, 10, 11 and 12 months. The degradation may take place in liquid, semi-solid, moist or dry conditions. The degradation is conveniently conducted aerobically, microaerobically and/or anaerobically. If desired, specific oxygen uptake rate can be used as a process control. The degradation can be conducted continuously, batch-wise, feed batch-wise or as any combination thereof.

In one embodiment the enzyme(s), micro-organism(s) or host cell(s) can be utilized for degrading hydrocarbon chains e.g. at a temperature below 100° C. such as 15-95° C., 30-95° C. or 40-80° C. (e.g. 50° C.). This indicates low energy need and therefore also moderate costs of the method.

In some embodiments of the invention an enzyme and/or enzymes (e.g. a combination of different enzymes) can produce material (e.g. degradation products (such as alkane) or modified material) for other enzymes or enzymes of other type(s) or micro-organisms to further degrade or modify said material (e.g. to fatty acids, PHA or diacids). On the other hand, in some embodiments of the invention a micro-organism, host cell, micro-organisms (e.g. a combination of different micro-organisms) or host cells can produce material (e.g. degradation products (such as alkane) or modified material) for micro-organisms of other type(s) or enzymes to further degrade or modify said material (e.g. to fatty acids, PHA or diacids).

In some embodiments of the present invention the micro-organisms or host cells are cultured under conditions (e.g. suitable conditions) in which the cultured micro-organism or host cell produces polypeptides, enzymes or compounds or interest (e.g. enzymes for degrading hydrocarbon chains). The micro-organisms or host cells can be cultivated in a medium containing appropriate carbon sources together with other optional ingredients selected from the group consisting of nitrogen or a source of nitrogen (such as amino acids, proteins, inorganic nitrogen sources such as nitrate, ammonia, urea or ammonium salts), yeast extract, peptone, minerals and vitamins, such as KH2PO4, Na2HPO, MgSO, CaCl2, FeCls, ZnSO, citric acid, MnSO, COCl2, CuSO, Na2MoO4, FeSO4, HsBO4, D-biotin, Ca-Pantothenate, nicotinic acid, myoinositol, thiamine, pyridoxine, p-amino benzoic acid. Suitable cultivation conditions, such as temperature, cell density, selection of nutrients, and the like are within the knowledge of a skilled person and can be selected to provide an economical process with the micro-organism in question. Temperatures may range from above the freezing temperature of the medium to about 50° C. or even higher, although the optimal temperature will depend somewhat on the particular micro-organism. In a specific embodiment the temperature is from about 25 to 35° C. The pH of the cultivation process may or may not be controlled to remain at a constant pH, but is usually between 3 and 9, depending on the production organism. Optimally the pH can be controlled e.g. to a constant pH of 7-8 (e.g. in the case of Escherichia coli) or to a constant pH of 5-6 (e.g. in the case of Yarrowia lipolytica). Suitable buffering agents include, for example, calcium hydroxide, calcium carbonate, sodium hydroxide, potassium hydroxide, potassium carbonate, hydrogen chloride, sodium carbonate, ammonium carbonate, ammonia, ammonium hydroxide and/or the like. In general, those buffering agents that have been used in conventional cultivation methods are also suitable here.

The micro-organisms or host cells can be normally separated from the culture medium after cultivation, before or after contacting with a hydrocarbon chain. The separated micro-organisms, host cells or a liquid (e.g. culture medium) comprising micro-organisms or host cells can be used for contacting hydrocarbon chains.

Polypeptides or enzymes can be secreted outside of the cells or they can stay in the cells. Therefore, the polypeptides or enzymes can be recovered from the cells or directly from the culture medium. In some embodiments both intracellular and extracellular polypeptides or enzymes are recovered. Prior to recovering, cells can be disrupted. Isolation and/or purification of polypeptides or enzymes can include one or more of the following: size exclusion, desalting, anion and cation exchange, based on affinity, removal of chemicals using solvents, extraction of the soluble proteinaceous material e.g. by using an alkaline medium (e.g. NaOH, Borate-based buffers or water is commonly used), isoelectric point-based or salt-based precipitation of proteins, centrifugation, and ultrafiltration. In one embodiment of the method, polypeptide or enzyme of the present invention, said polypeptide or enzyme is a purified or partly purified polypeptide or enzyme. If the polypeptide or enzyme is secreted outside of the cell it does not necessarily need to be purified.

Hydrocarbon chain(s) degrading enzymes can be expressed in any suitable host (cell). Examples of suitable host cells include but are not limited to cells of micro-organisms such as bacteria, yeast, fungi and filamentous fungi, as well as cells of plants and animals (such as mammals). Specific examples of host cells include but are not limited to Escherichia coli, Yarrowia lipolytica, Pichia pastoris, Trichoderma reesei, Aspergillus nidulans, Aspergillus niger, Bacillus licheniformis, Bacillus subtilis, Myceliophthora thermophila and Saccharomyces cerevisiae.

In one embodiment of the invention the micro-organism(s) or host cell(s) is(are) a bacterium or bacteria selected from the group comprising or consisting of Bacillus, Paenibacillus, Achromobacter, Acinetobacter, Alcanivorax, Aneurinibacillus, Arthrobacter, Brevibacillus, Chitinophaga, Citrobacter, Cupriavidus, Delftia, Enterobacter, Flavobacterium, Hyphomicrobium, Klebsiella, Kocuria, Leucobacter, Lysinibacillus, Macrococcus, Methylobacterium, Methylocella, Microbacterium, Micrococcus, Moraxella, Nesiotobacter, Nocardia, Ochrobactrum, Pantoea, Paracoccus, Pseudomonas, Rahnella, Ralstonia, Rhizobium, Rhodococcus, Serratia, Staphylococcus, Stenotrophomonas, Streptomyces, Vibrio, Virgibacillus and Xanthobacter; or the micro-organism(s) or host cell(s) is(are) a bacterium or bacteria selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Chitinophaga sp., Citrobacter amalonaticus, Cupriavidus necator, Delftia sp., Delftia tsuruhatensis, Enterobacter sp., Flavobacterium sp., Flavobacterium petrolei, Flavobacterium pectinovorum, Flavobacterium aquicola, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus lylae, Moraxella sp., Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Pseudomonas aeruginosa, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptomyces albogriseolus, Streptomyces badius, Streptomyces griseus, Streptomyces sp., Streptomyces viridosporus, Vibrio alginolyticus, Vibrio parahaemolyticus, Virgibacillus halodenitrificans, Xanthobacter autotrophicus, and Xanthobacter tagetidis, and any combination thereof.

Also, the micro-organism or host cell of the present invention can be used in a combination with any other micro-organism (simultaneously or consecutively), e.g. micro-organisms can be a population of different micro-organisms degrading different hydrocarbon chains or micro-organisms can be a combination of a bacterium and a fungus (to be used simultaneously or consecutively).

The inventors of the present disclosure have been able to isolate enzymes capable of degrading hydrocarbon chains from micro-organisms, and use said enzymes or micro-organisms for degrading hydrocarbon chains and/or producing degradation products of interest.

The present invention further relates to use of the enzyme, micro-organism, host cell, polynucleotide, expression vector or plasmid of the present invention or any combination thereof for degrading a hydrocarbon chain or hydrocarbon chains of different types.

Also, the present invention concerns a method of producing the enzyme of the present invention, wherein a recombinant micro-organism or host cell comprising the polynucleotide encoding the enzyme or fragment thereof of the present invention expresses or is allowed to express said enzyme or fragment thereof. For example, a vector or plasmid comprising the polynucleotide of interest can be transfected to a host cell, and the host cell can be used for expressing the enzyme of the present invention. In one embodiment the polynucleotide of interest is integrated into the genome of the host cell or the polynucleotide of interest is expressed from a vector or plasmid which is not integrated into the genome of the host cell. In one embodiment said expression of the enzyme can be controlled for example through inducible elements of promoters, vectors or plasmids.

As used in the present disclosure, the terms “polypeptide” and “protein” are used interchangeably to refer to polymers of amino acids of any length. As used herein “an enzyme” refers to a protein or polypeptide which is able to accelerate or catalyze (bio)chemical reactions.

As used herein “polynucleotide” refers to any polynucleotide, such as single or double-stranded DNA (genomic DNA or cDNA or synthetic DNA) or RNA (e.g. mRNA or synthetic RNA), comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

As used herein “isolated” enzymes, polypeptides or polynucleotides refer to enzymes, polypeptides or polynucleotides purified to a state beyond that in which they exist in cells. Isolated polypeptides, proteins or polynucleotides include e.g. substantially purified (e.g. purified to at least 10%, 20%, 30%, 40%, 50%, 60%, 70%, 80%, 90% purity) or pure enzymes, polypeptides or polynucleotides.

It is well known that a deletion, addition or substitution of one or a few amino acids of an amino acid sequence of an enzyme does not necessarily change the catalytic properties of said enzyme. Therefore, the invention also encompasses variants and fragments of the enzymes of the present invention or given amino acid sequences having the stipulated enzyme activity. The term “variant” as used herein refers to a sequence having minor changes in the amino acid sequence as compared to a given sequence. Such a variant may occur naturally e.g. as an allelic variant within the same strain, species or genus, or it may be generated by mutagenesis or other gene modification. It may comprise amino acid substitutions, deletions or insertions, but it still functions in substantially the same manner as the given enzymes, in particular it retains its catalytic function as an enzyme (e.g. capability to degrade a hydrocarbon chain). In one embodiment of the invention a fragment of the enzyme is an enzymatically active fragment or variant thereof.

A “fragment” of a given enzyme or polypeptide sequence means part of that sequence, e.g. a sequence that has been truncated at the N- and/or C-terminal end.

It may for example be the mature part of an enzyme or polypeptide comprising a signal sequence, or it may be only an enzymatically active fragment of the mature enzyme or polypeptide.

It will be obvious to a person skilled in the art that, as the technology advances, the inventive concept can be implemented in various ways. The invention and its embodiments are not limited to the examples described below but may vary within the scope of the claims.

### EXAMPLES

### Example 1. Expression of Bacillus licheniformis Metal-dependent Hydrolase in Escherichia coli

The gene encoding Bacillus licheniformis metal-dependent hydrolase (Genbank AKQ74356.1, SEQ ID NO: 2) amino acid was cloned from genomic Bacillus licheniformis DNA by PCR by using oligonucleotides oPlastBug-106 (ACAATTCCTCTAGAAATAATTTTGTTTAACTTTAAGAAGGAGATATATCCATGA AAGTGGCATATCATGGTCATTCAGTGG, SEQ ID NO: 18) and oPlastBug-107 (TTGTTAGCAGCCGGATCAAGCTGGGATTTAGGTGACACTATAGAATACTCTT ACTTAAATTCGATTGACTCACCGACCTCAA, SEQ ID NO: 19). The resulting DNA fragment containing coding region of the gene (SEQ ID NO: 1) was cloned into NcoI and HindIII digested Escherichia coli expression vector pBAT4 with Gibson assembly resulting in plasmid pPB030-1 and expressed in E. coli strain Shuffle T7 Express (New England Biolabs).

Plasmid pPB030-1 was expressed in E. coli Shuffle T7 Express grown at +37° C. in SB (30 g tryptone, 20 g yeast extract, 10 g MOPS (3-[N-morpholino]-propanesulfonic acid) per liter) media containing 100 μg/ml ampicillin. Protein expression was induced by the addition of 1 mM β-D-1-thiogalactopyranoside (IPTG), and induced cultures were further incubated at +37° C. for 24 hours. Cells were harvested by centrifugation (3184 g, 10 min RT), and supernatant was collected and stored at −80° C. until purification or was used directly in enzyme assays.

### Example 2. Purification of Expressed Hydrolases

The enzyme was purified using ion exchange (IEX) chromatography. The buffer of the culture filtrate was changed to 25 mM MES buffer, pH 6.7 using PD-10 desalting columns (Cytiva) and the sample was applied on an anion exchange DEAE sepharose fast flow 16/10 column (Cytiva) pre-equilibrated with 25 mM MES buffer, pH 6.7. The bound proteins were eluted with a 0-250 mM linear NaCl gradient for 20 column volumes (CV), where after the NaCl concentration was kept at 250 mM for 15 CV followed by a linear 250-1000 mM NaCl for 5 CV. Fractions containing the enzyme, as judged by SDS-PAGE analysis, were pooled, and concentrated using a Vivaspin sample concentrator (MWCO 5000; Sartorius, Germany). The purified enzyme was stored at −80° C.

The quality of purified protein was assessed by SDS-PAGE, to verify high enough (>85%) homogeneity of protein samples for upstream applications. Protein concentration was determined by Bio-Rad Bradford protein assay with BSA as standard by using the standard microplate assay. Samples were made in triplicate and were incubated for 15 min and A⁵⁹⁵ was measured with Varioskan Flash (Thermo Fischer).

### Example 3. Expression of Bacillus flexus, Bacillus subtilis and Bacillus cereus Metal-dependent Hydrolases in Escherichia coli

The gene encoding Bacillus flexus metal-dependent hydrolase (NCBI WP_035320333.1, SEQ ID NO: 6) amino acid was cloned from genomic Bacillus flexus DNA by PCR by using oligonucleotides oPlastBug-140 (ACAATTCCTCTAGAAATAATTTTGTTTAACTTTAAGAAGGAGATATATCCATGG TGCACATTTCTTATCACGGACACT, SEQ ID NO: 20) and oPlastBug-141 (TTGTTAGCAGCCGGATCAAGCTGGGATTTAGGTGACACTATAGAATACTCTT ACAAATCCAAACCTTCTCCAGGCTGTAAG, SEQ ID NO: 21). The resulting DNA fragment containing coding region of the gene (SEQ ID NO: 5) was cloned into NcoI and HindIII digested E. coli expression vector pBAT4 with Gibson assembly resulting in plasmid pPB046-2 and expressed in E. coli strain Shuffle T7 Express (New England Biolabs). The Plasmid pPB046-2 was expressed as described in Example 1. Supernatant sample was used directly in enzyme assays.

The gene encoding Bacillus subtilis metal-dependent hydrolase (Uniprot Q795U4, SEQ ID NO: 8) amino acid was cloned from genomic Bacillus subtilis DNA by PCR by using oligonucleotides oPlastBug-220(ACAATTCCTCTAGAAATAATTTTGTTTAACTTTAAGAAGGAGATATATCCA TGAAAGTGACATATCACGGACATTCTGTAATCAC, SEQ ID NO: 22) and oPlastBug-221 (TTGTTAGCAGCCGGATCAAGCTGGGATTTAGGTGACACTATAGAATACT CTTAAAGCTCGATCGTCTCACCGACCG, SEQ ID NO: 23). The resulting DNA fragment containing coding region of the gene (SEQ ID NO: 7) was cloned into NcoI and HindIII digested E. coli expression vector pBAT4 with Gibson assembly resulting in plasmid pPB075-1 and expressed in E. coli strain Shuffle T7 Express (New England Biolabs). The Plasmid pPB075-1 was expressed as described in Example 1. Supernatant sample was used directly in enzyme assays.

The gene encoding Bacillus cereus metal-dependent hydrolase (SEQ ID NO: 4) amino acid was cloned from genomic Bacillus cereus DNA by PCR by using oligonucleotides oPlastBug-110 (ACAATTCCTCTAGAAATAATTTTGTTTAACTTTAAGAAGGAGATATATCCATGA AAGTATCTTATCATGGACATTCAGTTGTGAAA, SEQ ID NO: 24) and oPlastBug-111 (TTGTTAGCAGCCGGATCAAGCTGGGATTTAGGTGACACTATAGAATAC TCCTATAGTGTAATACTTTCTCCAGCTTCTAATACTTTCCC, SEQ ID NO: 25). The resulting DNA fragment containing coding region of the gene (SEQ ID NO: 3) was cloned into NcoI and HindIII digested E. coli expression vector pBAT4 with Gibson assembly resulting in plasmid pPB031-5 and expressed in E. coli strain Shuffle T7 Express (New England Biolabs). The Plasmid pPB031-5 was expressed as described in Example 1. Supernatant sample was used directly in enzyme assays.

### Example 4. Metal-dependent Hydrolase Activity With Bacillus licheniformis, Bacillus flexus, Bacillus subtilis and Bacillus cereus enzymes

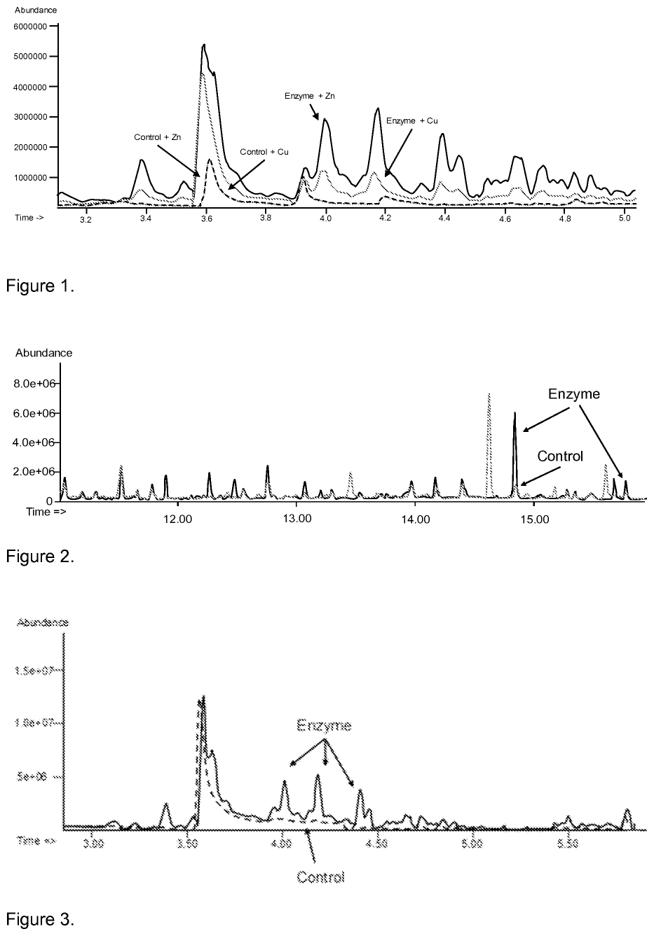

Enzyme assay with Bacillus licheniformis enzyme was carried out as follows: One ml of 50 mM Bicine, pH 9.0 with 1 mM ZnSO4 or 1 mM CuCl₂ and polyethylene powder (average MW ˜4000 dalton, Sigma-Aldrich) was incubated with 50 μl of E. coli supernatant samples from Example 1 at +50° C. for 90 hours. As a control supernatant from the culture with E. coli strain having empty plasmid (pBAT4) was used. After incubation SPME GC-MS run was carried out with liquid fraction as described in Example 5. Results from the GC-MS run are shown in FIG. 1. With enzyme samples with Zn and Cu several peaks appeared which were missing from control samples. These peaks presented alkanes like compounds.

Enzyme assay with Bacillus licheniformis enzyme with LDPE film was carried out as follows: One ml of 150 mM Bicine, pH 8.0 with 1 mM ZnSO4 and LDPE film (Thickness 0.23 mm, additive free polymer, biaxially oriented, Goodfellow) was incubated with 100 μl of E. coli supernatant samples from Example 1 at +50° C. for 46 hours. As a control supernatant from the culture with E. coli strain having empty plasmid (pBAT4) was used. After incubation SPME GC-MS run was carried out with liquid fraction as described in Example 5. Results from the GC-MS run are shown in FIG. 2. With enzyme samples with Zn several peaks appeared which were missing from control samples. These peaks presented alkanes like compounds.

Enzyme assay with Bacillus flexus enzyme was carried out as follows: One millilitre of 50 mM HEPES, pH 8.0 with polyethylene powder (˜4000 Da, Sigma-Aldrich) was incubated with 50 μl of E. coli supernatant samples from Example 3 at +30° C. for 94 hours. SPME GC-MS run was carried out with liquid fraction as described in Example 5. Results from the GC-MS run is shown in FIG. 3. With samples having enzyme without added metals several peaks appeared which were not seen when control sample (empty plasmid in E. coli) was used in the reaction. These peaks presented alkanes like compounds.

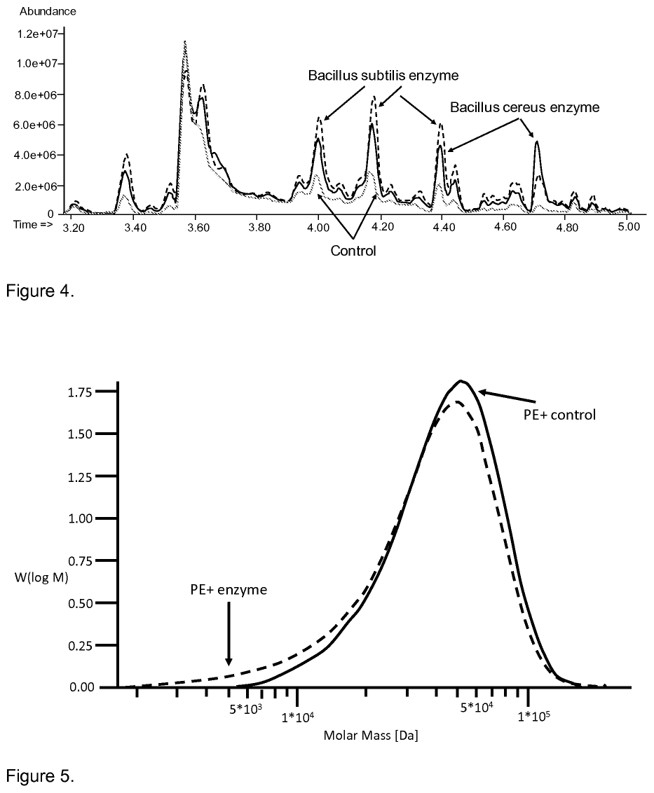

Enzyme assay with Bacillus subtilis and Bacillus cereus enzymes was carried out as follows: One millilitre of 50 mM HEPES, pH 8.0 with 1 mM of ZnSO4 and polyethylene powder (˜4000 Da, Sigma-Aldrich) was incubated with 50 μl of E. coli supernatant samples from Example 3 at +30° C. for 72 hours. SPME GC-MS run was carried out with liquid fraction as described in Example 5. Results from the GC-MS run is shown in FIG. 4. With samples having enzyme with Zn metal several peaks appeared which were not seen when control sample (empty plasmid in E. coli) was used in the reaction. These peaks presented alkanes like compounds.

### Example 5. Gas Chromatography-mass Spectrometry (GC-MS) Analysis of Volatile Degradation Products of Polyethylene With Metal Dependent Hydrolases

Samples from examples 4, 9 and 10 were transferred to GC-MS vials and run directly with a Headspace-SPME-GC-MS method as described below. The samples were incubated at 60° C. for 1 min and for the fiber (Supelco DVB/Car/PDMS Stableflex 2 cm) the extraction and desorption times were 30 min and 480 s, respectively. The runs were performed on Agilent GC-MS equipped with an HP-FFAP (25 m×200 μm×0.3 μm) column and helium was used as a carrier gas. The injector temperature was 250° C., and a splitless injection mode was used. The oven temperature was 40° C. for 3 min, increased to 240° C. at 15° C./min and kept at 240° C. for 9 min. The detected mass range was 30-400 m/z. Identification of the volatile compounds was based on NIST08 MS library. Results from GC-MS analysis are described in Examples 4, 9 and 10.

### Example 6. Heat-GPC Analysis Results With Hydrolase

Enzyme reaction containing 50 μg of purified B. licheniformis metal-dependent hydrolase from Example 2 was incubated in 50 mM HEPES, pH 8.0, 1 mM ZnSO4 with 16 kDa polyethylene (from PSS Polymer Standards Service GmbH) 6 days at +50° C. In control reaction enzyme was replaced with water. After incubation liquid fraction was removed and solid polyethylene fraction was dried at +50° C. Dried polyethylene fraction was analysed with heat GPC carried out by PSS Polymer Standards Service GmbH: Dried samples were solved to 1,2,4-trichlorbenzol at +160 C for one hour. The sample solution was filtered through a HT-filter unit and 200 ul of sample (1 or 3 g/l) was injected. Samples were run with 1.00 ml/min at +160° C. with 1,2,4-trichlorbenzol by using precolumn PSS POLEFIN 20 μm, Guard, ID 8×50 mm and four columns of PSS POLEFIN linear XL, 20 μm, ID 8×300 mm. Detection was carried out with IR4 detector. Several polystyrene standards with different molecular weight were measured first in order to get a calibration curve. The calculation of the average molecular weights and the molecular weight distribution of the samples was done by the so called slice by slice method based on the PS-calibration by using PSS WinGPC UniChrom Version 8.33. Values detected with polystyrene can be converted to polyethylene by using so called Mark-Houwink equation parameter (PE values are 2-2.4 times lower). Results from heat GPC are shown in Table 1 and FIG. 5.

In heat GPC 23% reduction in Mn (number average molecular weight), 8% in MW (weight average molecular weight) and 5% in MP (Molar mass at the peak maximum) could be detected. Additionally, PDI (polydispersity index) increased 20% and area 16%. These results indicate that degradation of polyethylene has happened in the sample with enzyme. Also amount of shorter polyethylene polymers have increased during enzyme treatment (FIG. 5).

TABLE 1
Heat GPC results with Bacillus licheniformis
enzyme. Values are based on polystyrene standard.

| Sample | Mn (Da) | Mw (Da) | PDI | Mp (Da) | Area |
|---|---|---|---|---|---|
| Control | 35500 | 48200 | 1.36 | 52800 | 0.688860 |
| Enzyme | 27200 | 44400 | 1.63 | 50400 | 0.797260 |
| Change | −23% | −8% | +20% | −5% | +16% |

### Example 7. Characterisation of Amino Acid Sequence Motifs of Polyethylene Degrading Metal-dependent Hydrolases

A sequence search based on HMMER (Robert D. Finn, Jody Clements, Sean R. Eddy (2011) HMMER web server: interactive sequence similarity searching. Nucleic Acids Research, Volume 39, Issue suppl_2, 1 July 2011, Pages W29-W37, https://doi.org/10.1093/nar/gkr367) was done using the amino acid sequences of SEQ ID NOs: 2, 4, 6, 8, 10 and 12. The HMMER search was carried out against the UNIPROT. The results were filtered based on the e-value. Several hundreds of sequences were identified. Among these sequences amino acid sequences originating from species which have been shown to degrade polyethylene were collected (SEQ ID NOs 2, 4, 6, 8, 10, 12, 26-66) and used in multiple sequence alignment carried out with CLUSTAW (https://www.genome.jp/tools-bin/clustalw) with default parameters.

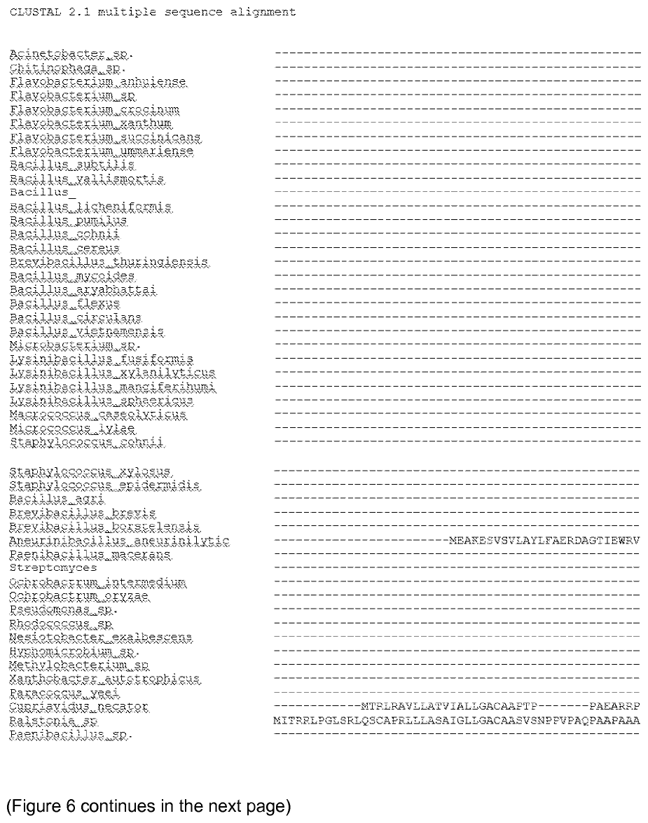

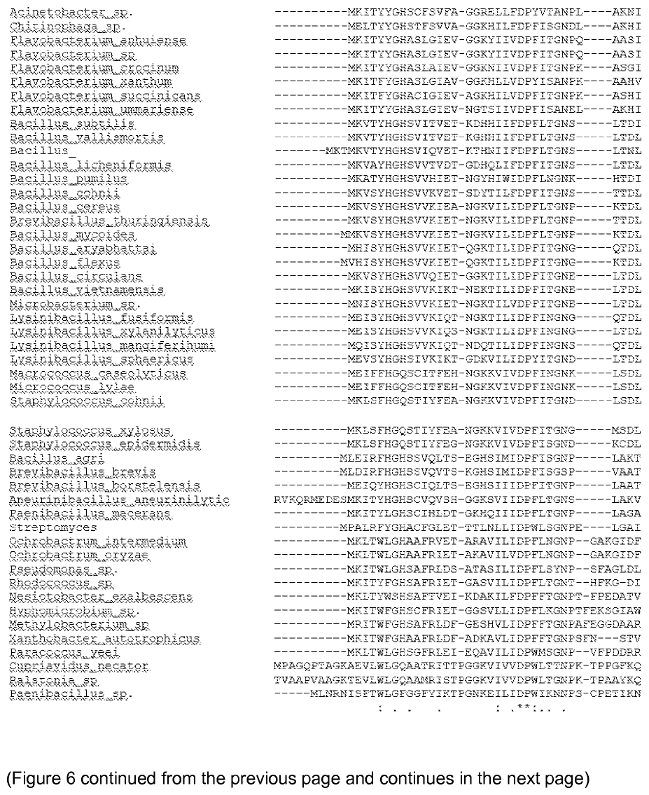

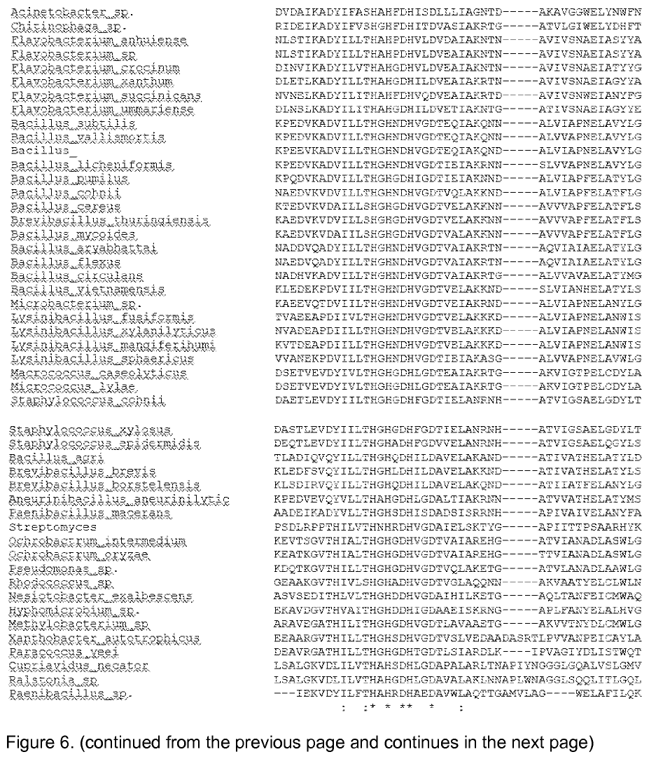

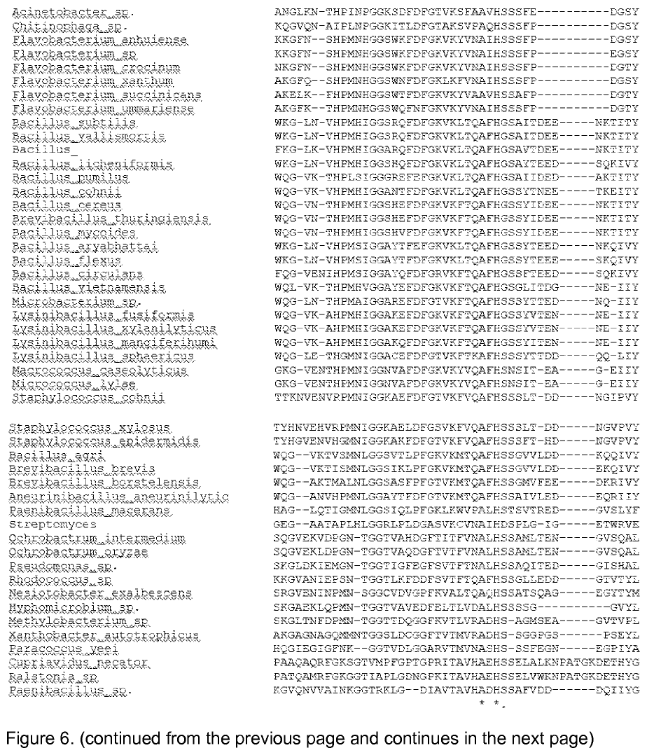

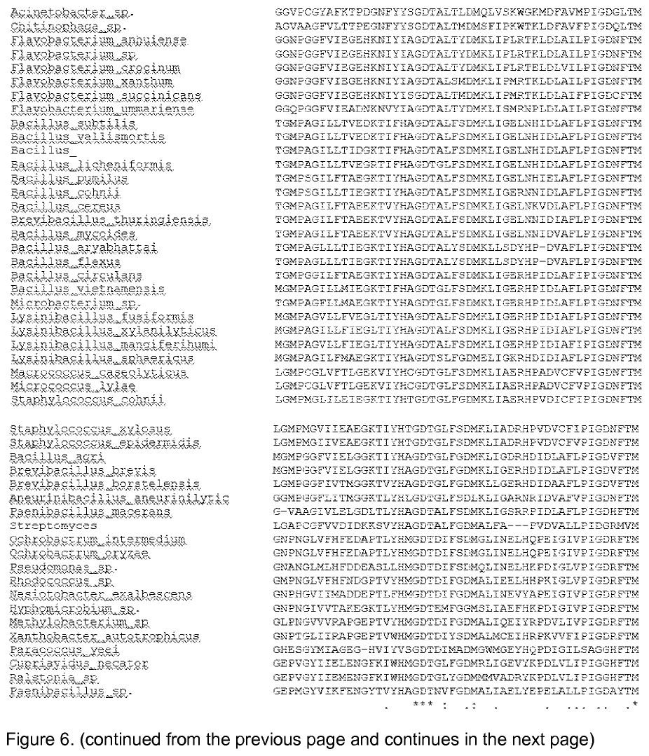

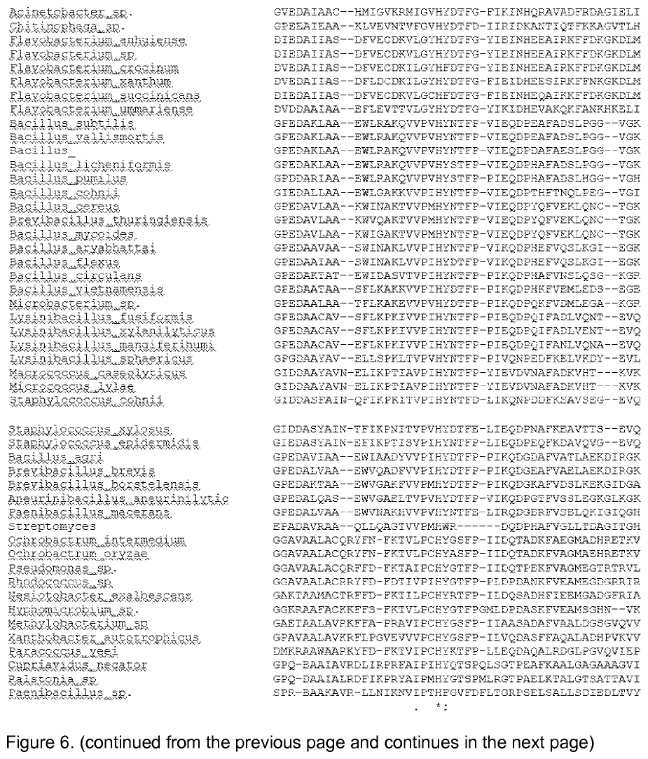

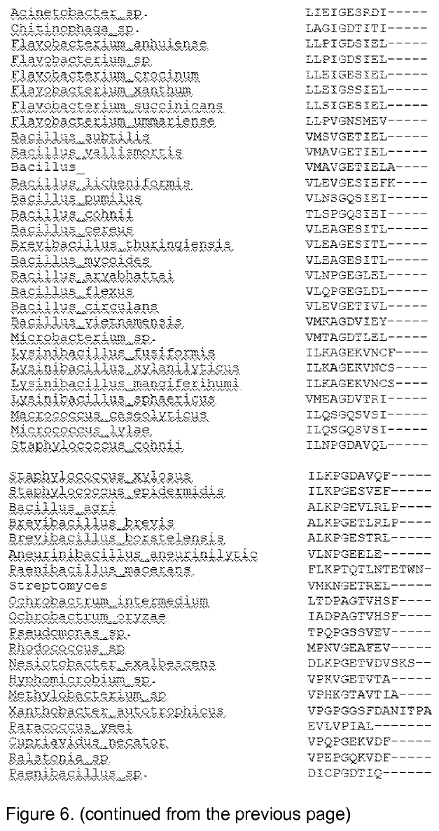

In the alignment several consensus amino acids could be detected (based on Bacillus licheniformis amino acid position): Asp23, Pro24, His48, His50, Asp52, His53, Asp56, Ala106, His108, Gly142, Asp143, Thr144, Met172, His193 (see FIG. 6).

To confirm the existence and position of consensus amino acids in a specific enzyme corresponding amino acid sequence was compared to B. licheniformis metal dependent hydrolase (SEQ ID NO: 2) by carrying out pairwise alignment with ClustalW default parameters by using Geneious 10.2.6 programme. In FIG. 7 is an example of pairwise alignment between Ralstonia sp metal dependent hydrolase (SEQ ID NO: 60) and B. licheniformis metal dependent hydrolase (SEQ ID NO: 2). Even these amino acid sequences have only 23% identity between each other abovementioned consensus amino acids could be detected and their position in Ralstonia sp. amino acid sequence identified (mark in bold).

### Example 8. Expression of Brevibacillus borstelensis and Micrococcus lylae Metal-dependent Hydrolase in Escherichia coli

The genes encoding Brevibacillus borstelensis (Genbank EMT53158.1, Sequence N:O 15) and Micrococcus lylae (SEQ ID NO: 17) metal-dependent hydrolase amino acids were commercially (Genscript) synthetized with codon optimization for expression in Escherichia coli cells (SEQ ID NOs: 14 and 16, respectively). NcoI and HindIII restriction sites were included at 5′ and 3′ ends of construct for restriction digestion cloning. The constructs were cloned into E. coli expression vector pBAT4 by restriction digestion cloning and expressed in E. coli strain Shuffle T7 Express (New England Biolabs). Enzymes were produced as described in Example 1.

### Example 9. Metal-dependent Hydrolase Activity With Brevibacillus borstelensis and Micrococcus lylae Enzymes

Enzyme assay with Brevibacillus borstelensis enzyme was carried out as follows: One millilitre of 50 mM HEPES, pH 8.0 with 1 mM of NiSO4 with polyethylene powder (˜4000 Da, Sigma-Aldrich) was incubated 50 μl of E. coli supernatant samples from Example 8 at +50° C. for 53 hours. SPME GC-MS run was carried out with liquid fraction as described in Example 5. Results from the GC-MS run is shown in FIG. 8. With samples having enzyme with Ni metals several peaks appeared which were not seen when control sample (empty plasmid in E. coli) was used in the reaction. These peaks presented different short chain hydrocarbon compounds

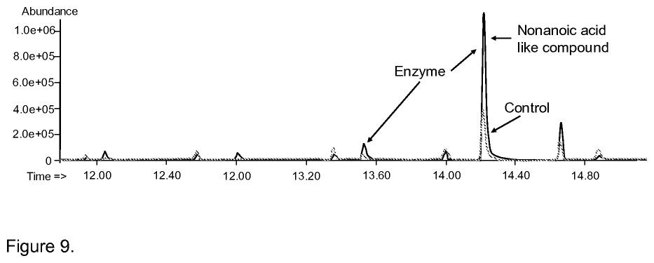

Enzyme assay with Micrococcus lylae enzyme was carried out as follows: One millilitre of 50 mM HEPES, pH 8.0 with 1 mM ZnSO4 with polyethylene powder (˜4000 Da, Sigma-Aldrich) was incubated 50 μl of E. coli supernatant samples from Example 8 at +30° C. for 53 hours. SPME GC-MS run was carried out with liquid fraction as described in Example 5. Results from the GC-MS run is shown in FIG. 9. With samples having enzyme with Zn metals several peaks appeared which were not seen when control sample (empty plasmid in E. coli) was used in the reaction. These peaks presented different short chain hydrocarbon compounds

### Example 10. Expression of Bacillus licheniformis Metal-dependent Hydrolase in Yarrowia lipolytica

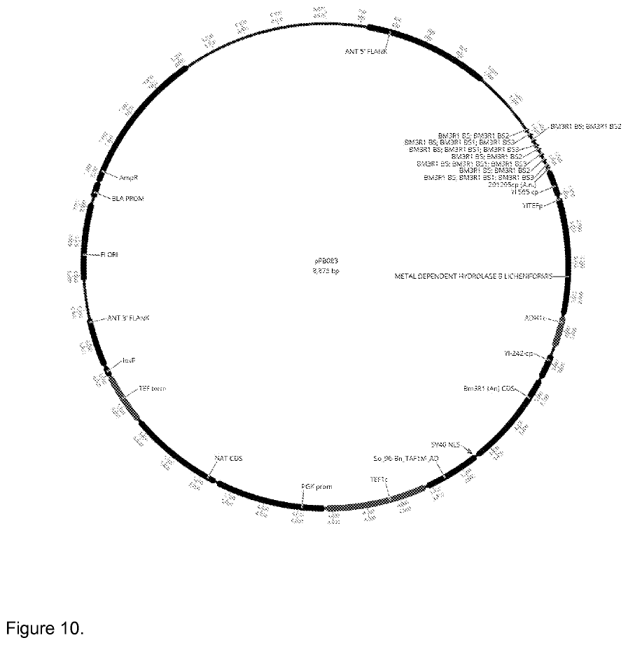

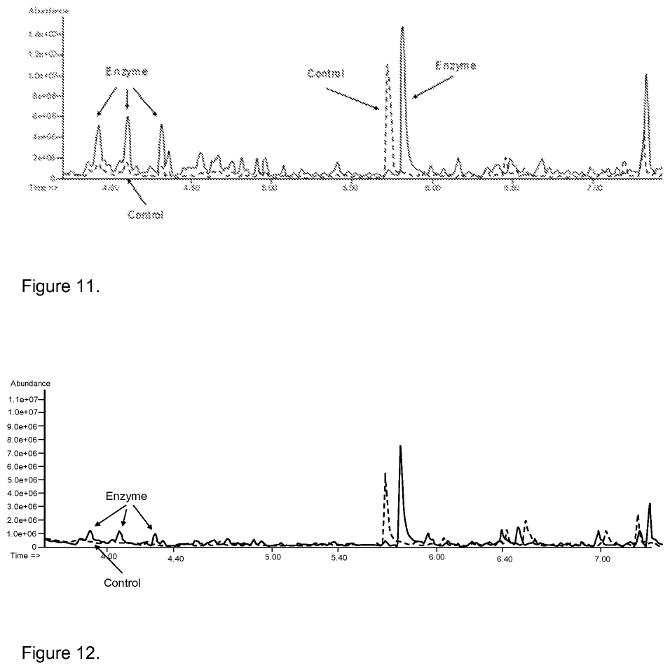

The gene encoding Bacillus licheniformis metal-dependent hydrolase (Genbank AKQ74356.1, SEQ ID NO: 2) amino acid was commercially (Genscript) synthetized with codon optimization for expression in Yarrowia lipolytica cells (SEQ ID NO: 13). PacI and BglII restriction sites were included at 5′ and 3′ ends of construct for restriction digestion cloning. The constructs were cloned into Yarrowia lipolytica integration cassette plasmid B11157 digested with PacI and BclI. B11157 plasmid contains flanks to ANT1 gene and SES promoter (SES promoter described in Rantasalo et al 2018. Nucleic Acids Research, Volume 46, issue 18, 12 October 2018, Page e111, https://doi.org/10.1093/nar/gky558). The resulting plasmid was named as pPB083 (FIG. 10). NotI digested integration fragment was transformed into VTT-C-00365 Yarrowia lipolytica strain (VTTCC) with Frozen-EZ yeast transformation kit. After transformation single colonies were cultivated in 3.5 ml of synthetic complete medium containing 4% glucose in 24 well plates. Wild type Yarrowia lipolytica VTT-C-00365 were cultivated as a control. After 3 days incubation at +30° C. with 200 rpm shaking cultures were centrifuged (3184 g, 10 min RT) and supernatant and pellet samples were separated. Pellet samples were homogenies as follows: Pellet was suspended into 1 ml of 50 mM HEPES, pH 8.0 and homogenised with glass beads with Precellys homogenizator. After homogenisation samples were centrifuged (20817 g, 27 min at +4 C) and cell extracts were collected. Enzyme activity measurements with cell extracts and supernatant samples from cultivations were carried out as follows: 100 μl of supernatant sample or 750 μl of cell extract was incubated in 50 mM HEPES pH 8.0 with 1 mM ZnSO4 and PE powder (˜4000 Da, Sigma-Aldrich) at +50° C. for 3.5 days (for cell extract samples) and 7 days (for supernatant samples). After enzyme reaction SPME GCMS was run as described in Example 5. In GCMS analysis several peaks could be detected with Y. lipolytica strains expressing B. licheniformis metal dependent hydrolase both with cell extract (FIG. 11) and supernatant (FIG. 12) samples which could not be detected with control samples (wild type C-00365 Yarrowia lipolytica). These detected peaks were alkane like compounds like seen in Example 4. This result indicates that B. licheniformis metal-dependent hydrolase can be expressed functionally in Y. lipolytica.

### Example 11. Localising Consensus Amino Acids into Enzymes 2D and 3D Structure

Two-dimensional and 3 D structures of Bacillus licheniformis metal dependent hydrolase (SEQ ID N:O 2) was constructed with Phyre2 protein homology/analogy recognition engine V 2.0 (www.sbg.bio.ic.ac.uk/phyre2/html/page.cgi?id=index) with default parameters. The predicted alfa helixes and beta sheets were localised together with identified consensus amino acids from Example 7 into amino acid sequence shown in FIG. 13. The amino acids which in predicted 3D structure were critical to metal binding, right protein structure and activity were identified. These amino acids were in loop area (Asp23, His48, His50, Asp52, His53, Asp143) and in beta sheet 7 (His108).

1. A method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises amino acids D23, H48, H50, D52, H53, H108 and D143 corresponding to the amino acid positions presented in SEQ ID NO: 2 wherein the enzyme is optionally genetically modified.

2. An isolated enzyme or a fragment thereof comprising amino acids D23, H48, H50, D52, H53, H108 and D143 corresponding to the amino acid positions presented in SEQ ID NO: 2, wherein said enzyme or fragment is capable of degrading a hydrocarbon chain, wherein the enzyme is optionally genetically modified.

3. A micro-organism or a host cell comprising an enzyme or a fragment thereof comprising amino acids D23, H48, H50, D52, H53, H108 and D143 corresponding to the amino acid positions presented in SEQ ID NO: 2, wherein said enzyme or fragment is capable of degrading a hydrocarbon chain, wherein the micro-organism or the host cell is optionally genetically modified.

4. The method according to claim 1, wherein the enzyme or fragment thereof comprises amino acids D23, H48, H50, D52, H53, H108 and D143 corresponding to the amino acid positions presented in SEQ ID NO: 2 and at least one of the amino acids selected from the group consisting of the amino acids P24, D56, A106, G142, T144, M172 and H193 corresponding to the amino acid positions presented in SEQ ID NO: 2.

5. The method of claim 1, wherein the enzyme has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, and the enzyme optionally originates from or is an enzyme of a bacterium selected from the group comprising or consisting of Bacillus, Paenibacillus, Achromobacter, Acinetobacter, Alcanivorax, Aneurinibacillus, Arthrobacter, Brevibacillus, Chitinophaga, Citrobacter, Cupriavidus, Delftia, Enterobacter, Flavobacterium, Hyphomicrobium, Klebsiella, Kocuria, Leucobacter, Lysinibacillus, Macrococcus, Methylobacterium, Methylocella, Microbacterium, Micrococcus, Moraxella, Nesiotobacter, Nocardia, Ochrobactrum, Pantoea, Paracoccus, Pseudomonas, Rahnella, Ralstonia, Rhizobium, Rhodococcus, Serratia, Staphylococcus, Stenotrophomonas, Streptomyces, Vibrio, Virgibacillus and Xanthobacter; or the enzyme is an enzyme of a bacterium selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Chitinophaga sp., Citrobacter amalonaticus, Cupriavidus necator, Delftia sp., Delftia tsuruhatensis, Enterobacter sp., Flavobacterium sp., Flavobacterium petrolei, Flavobacterium pectinovorum, Flavobacterium aquicola, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus lylae, Moraxella sp., Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Pseudomonas aeruginosa, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptomyces albogriseolus, Streptomyces badius, Streptomyces griseus, Streptomyces sp., Streptomyces viridosporus, Vibrio alginolyticus, Vibrio parahaemolyticus, Virgibacillus halodenitrificans, Xanthobacter autotrophicus, and Xanthobacter tagetidis.

6. The isolated enzyme or fragment thereof, according to claim 2, wherein the enzyme has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, and wherein the enzyme originates from or is an enzyme of a bacterium selected from the group comprising or consisting of Bacillus, Paenibacillus, Achromobacter, Acinetobacter, Alcanivorax, Aneurinibacillus, Arthrobacter, Brevibacillus, Chitinophaga, Citrobacter, Cupriavidus, Delftia, Enterobacter, Flavobacterium, Hyphomicrobium, Klebsiella, Kocuria, Leucobacter, Lysinibacillus, Macrococcus, Methylobacterium, Methylocella, Microbacterium, Micrococcus, Moraxella, Nesiotobacter, Nocardia, Ochrobactrum, Pantoea, Paracoccus, Pseudomonas, Rahnella, Ralstonia, Rhizobium, Rhodococcus, Serratia, Staphylococcus, Stenotrophomonas, Streptomyces, Vibrio, Virgibacillus and Xanthobacter; or the enzyme is an enzyme of a bacterium selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Chitinophaga sp., Citrobacter amalonaticus, Cupriavidus necator, Delftia sp., Delftia tsuruhatensis, Enterobacter sp., Flavobacterium sp., Flavobacterium petrolei, Flavobacterium pectinovorum, Flavobacterium aquicola, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus lylae, Moraxella sp., Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Pseudomonas aeruginosa, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptomyces albogriseolus, Streptomyces badius, Streptomyces griseus, Streptomyces sp., Streptomyces viridosporus, Vibrio alginolyticus, Vibrio parahaemolyticus, Virgibacillus halodenitrificans, Xanthobacter autotrophicus, and Xanthobacter tagetidis.

7. (canceled)

8. The method according to claim 1, wherein the hydrocarbon chain is a hydrocarbon chain of a synthetic polymer, alkane, alkene, alkyne, cycloalkane, alkadiene, ketone, fatty acid, alcohol, aldehyde, polyolefin, polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low density polyethylene (VLDPE), or any combination thereof, and wherein the length of the hydrocarbon chain is optionally at least C20, C22, C24, C26, C28, C30, C32, C34, C36, C38, C40, C45, C50, C60, C70, C80, C90, C100, C150, C200, C250, C300, C350, C400, C450 or C500.

9. The isolated enzyme or fragment thereof according to claim 2, wherein the hydrocarbon chain is a hydrocarbon chain of a synthetic polymer, alkane, alkene, alkyne, cycloalkane, alkadiene, ketone, fatty acid, alcohol, aldehyde, polyolefin, polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low density polyethylene (VLDPE), or any combination thereof, and the length of the hydrocarbon chain is optionally at least C20, C22, C24, C26, C28, C30, C32, C34, C36, C38, C40, C45, C50, C60, C70, C80, C90, C100, C150, C200, C250, C300, C350, C400, C450 or C500.

10. (canceled)

11. (canceled)

12. (canceled)

13. The method of claim 1, wherein the enzyme or fragment thereof has an increased ability to degrade the hydrocarbon chain compared to the corresponding unmodified enzyme or fragment thereof, and wherein the enzyme or fragment thereof optionally does not comprise a detectable signal sequence and is secreted out of the cell which produces the enzyme or fragment thereof.

14. (canceled)

15. (canceled)

16. The host cell of claim 3, wherein the host cell is selected from the group consisting of Escherichia coli, Yarrowia lipolytica, Pichia pastoris, Trichoderma reesei, Aspergillus nidulans, Aspergillus niger, Bacillus licheniformis, Bacillus subtilis, Myceliophthora thermophila and Saccharomyces cerevisiae.

17. A polynucleotide encoding the enzyme or fragment thereof of claim 2.

18. An expression vector or plasmid comprising the polynucleotide of claim 17.

19. (canceled)

20. A method of producing the enzyme or fragment thereof of claim 2, wherein a recombinant micro-organism or host cell comprising a polynucleotide encoding the enzyme or fragment thereof is allowed to express said enzyme or fragment thereof.

21. The isolated enzyme or fragment thereof according to claim 2, wherein the enzyme or fragment thereof comprises amino acids D23, H48, H50, D52, H53, H108 and D143 corresponding to the amino acid positions presented in SEQ ID NO: 2 and at least one of the amino acids selected from the group consisting of the amino acids P24, D56, A106, G142, T144, M172 and H193 corresponding to the amino acid positions presented in SEQ ID NO: 2.

22. The micro-organism or host cell according to claim 3, wherein the enzyme or fragment thereof comprises amino acids D23, H48, H50, D52, H53, H108 and D143 corresponding to the amino acid positions presented in SEQ ID NO: 2 and at least one of the amino acids selected from the group consisting the amino acids P24, D56, A106, G142, T144, M172 and H193 corresponding to the amino acid positions presented in SEQ ID NO: 2.

23. The micro-organism or host cell-according to claim 3, wherein the enzyme has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, 10, 12, 15, or 17, and wherein the enzyme originates from or is an enzyme of a bacterium selected from the group comprising or consisting of Bacillus, Paenibacillus, Achromobacter, Acinetobacter, Alcanivorax, Aneurinibacillus, Arthrobacter, Brevibacillus, Chitinophaga, Citrobacter, Cupriavidus, Delftia, Enterobacter, Flavobacterium, Hyphomicrobium, Klebsiella, Kocuria, Leucobacter, Lysinibacillus, Macrococcus, Methylobacterium, Methylocella, Microbacterium, Micrococcus, Moraxella, Nesiotobacter, Nocardia, Ochrobactrum, Pantoea, Paracoccus, Pseudomonas, Rahnella, Ralstonia, Rhizobium, Rhodococcus, Serratia, Staphylococcus, Stenotrophomonas, Streptomyces, Vibrio, Virgibacillus and Xanthobacter; or
the enzyme is an enzyme of a bacterium selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Chitinophaga sp., Citrobacter amalonaticus, Cupriavidus necator, Delftia sp., Delftia tsuruhatensis, Enterobacter sp., Flavobacterium sp., Flavobacterium petrolei, Flavobacterium pectinovorum, Flavobacterium aquicola, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus lylae, Moraxella sp., Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Pseudomonas aeruginosa, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Staphylococcus epidermidis, Staphylococcus cohnii, Staphylococcus xylosus, Stenotrophomonas humi, Stenotrophomonas maltophilia, Stenotrophomonas panacihumi, Stenotrophomonas sp., Streptomyces albogriseolus, Streptomyces badius, Streptomyces griseus, Streptomyces sp., Streptomyces viridosporus, Vibrio alginolyticus, Vibrio parahaemolyticus, Virgibacillus halodenitrificans, Xanthobacter autotrophicus, and Xanthobacter tagetidis.

24. The micro-organism or host cell according to claim 3, wherein the hydrocarbon chain is a hydrocarbon chain of a synthetic polymer, alkane, alkene, alkyne, cycloalkane, alkadiene, ketone, fatty acid, alcohol, aldehyde, polyolefin, polyethylene (PE), cross-linked polyethylene (PEX or XLPE), ultra-high molecular weight polyethylene (UHMWPE), high-density polyethylene (HDPE), medium-density polyethylene (HDPE), linear low-density polyethylene (LLDPE), low density polyethylene (LDPE), very low density polyethylene (VLDPE), or any combination thereof, and the length of the hydrocarbon chain is optionally at least C20, C22, C24, C26, C28, C30, C32, C34, C36, C38, C40, C45, C50, C60, C70, C80, C90, C100, C150, C200, C250, C300, C350, C400, C450 or C500.

25. The isolated enzyme or fragment thereof fragment of claim 2, wherein at least one or more degradation products selected from the group consisting of an alkane, alkene, alkyne, cycloalkane, alkadiene, ketone, fatty acid, alcohol, aldehyde, epoxy, benzene, styrene, diacid, 2-decanone, 2-dodecanone, 2-tetradecanone, 2-hexadecanone, 2-heptadecanone and 2-dotriacontanone are obtained or obtainable by the degradation of the hydrocarbon chain.

26. The micro-organism or host cell of claim 3, wherein at least one or more degradation products selected from the group consisting of an alkane, alkene, alkyne, cycloalkane, alkadiene, ketone, fatty acid, alcohol, aldehyde, epoxy, benzene, styrene, diacid, 2-decanone, 2-dodecanone, 2-tetradecanone, 2-hexadecanone, 2-heptadecanone and 2-dotriacontanone are obtained or obtainable by the degradation of the hydrocarbon chain.

27. The micro-organism or host cell of claim 3, wherein the enzyme or a fragment thereof has an increased ability to degrade the hydrocarbon chain compared to the corresponding unmodified enzyme, and wherein the enzyme or fragment thereof does not comprise a detectable signal sequence and is secreted out of the cell which produces the enzyme or fragment thereof.


SEQUENCE LISTING (66 sequences)

SEQ ID NO: 1 - DNA, 687 bp, Bacillus licheniformis
atgaaagtggcatatcatggtcattcagtggtaacagttgatacgggagatcatcagttgatcttcgatccgtttattacaggaaactcattaaccgatttaaagcccgaagatgtcaaagcggacgtcatcttgctgactcacggacataacgatcatgtcggcgataccatcgaaatcgccaagcgaaacaattcgctcgttgtagctccgaatgaactcgcggtctatctaggatggaaaggcctgaatgtacatccgatgcacatcggcggctcccaccagttcgattttggaaaagtcaagctgacgcaggctttccacggatcggcttatacggaagaagacagccaaaagatcgtctataccggcatgccggcggggatccttctgactgtggaaggaaggacgatttttcatgccggcgacaccgggcttttctcagatatgaagctgatcggtgaattaaatcatatcgaccttgccttcttgccgatcggcgataattttacgatgggccctgaggacgccaaactggccgcagaatggctcagagcgaaacaggttgtcccggttcactactcgacgttccccgtcatcgaacaggatccgcatgcatttgcggacagccttcctggaggcgtcggaaaagtgcttgaggtcggtgagtcaatcgaatttaagtaa

SEQ ID NO: 2 - protein, 228 aa, Bacillus licheniformis
MKVAYHGHSVVTVDTGDHQLIFDPFITGNSLTDLKPEDVKADVILLTHGHNDHVGDTIEIAKRNNSLVVAPNELAVYLGWKGLNVHPMHIGGSHQFDFGKVKLTQAFHGSAYTEEDSQKIVYTGMPAGILLTVEGRTIFHAGDTGLFSDMKLIGELNHIDLAFLPIGDNFTMGPEDAKLAAEWLRAKQVVPVHYSTFPVIEQDPHAFADSLPGGVGKVLEVGESIEFK

SEQ ID NO: 3 - DNA, 684 bp, Bacillus cereus
atgaaagtatcttatcatggacattcagttgtgaagattgaaacgaatggaaaagtgattgtaattgatccatttttaacgggaaatccgaaaacagatttaaaggctgaagatgtaaaagtagatgcgattcttttatcgcacggacatggtgatcatgttggagatacagtagagcttgcgaagaaaaataatgcagttgtagtggcgccatttgaactagcgacatttttaagttggcaaggtgtaaaaacacatccgatgcatattggtggttcacatgaatttgacttcggaaaagtgaagtttacacaagcattccacggctctagttatattgatgaagaaaataagacgattacatatacaggtatgccagcgggtattttatttacagcagaagaaaaaactgtgtatcatgcaggggatactgctctattctctgatatgaaattaattggggaactaaataacattgatgtagcatttttaccaattggtgataatttcacaatgggaccagaagatgctgttttagcagcaaaatgggttcaagcgaaaactgttgtaccgatgcattacaatacgttcccggttattgaacaagatccatatcaatttgtagaaaagctacaaaattgtacagggaaagtattagaagctggagaaagtattacactatag

SEQ ID NO: 4 - protein, 227 aa, Bacillus cereus
MKVSYHGHSVVKIETNGKVIVIDPFLTGNPKTDLKAEDVKVDAILLSHGHGDHVGDTVELAKKNNAVVVAPFELATFLSWQGVKTHPMHIGGSHEFDFGKVKFTQAFHGSSYIDEENKTITYTGMPAGILFTAEEKTVYHAGDTALFSDMKLIGELNNIDVAFLPIGDNFTMGPEDAVLAAKWVQAKTVVPMHYNTFPVIEQDPYQFVEKLQNCTGKVLEAGESITL

SEQ ID NO: 5 - DNA, 684 bp, Bacillus flexus
atggtgcacatttcttatcacggacactctgttgtgaaaattgaaacgcaagggaaaacaattttaatcgatccgtttattacaggaaatggaaaaactgatttaaatgcagaggatgtacaagcagactatatcttactaacgcacggtcataacgatcacgtaggagatacagtagccattgcaaaacgaacaaatgcgcaggttattgctattgctgagctcgctacatatttaggatggaaaggtttaaatgttcatcccatgagcattggaggagcttatacgtttgattttggtaaagttaaactcacacaagcttttcatggatcaagctatacggaagaagaaagcaagcaaattgtttatacgggcatgccagcaggcttgctgctaacaattgaaggaaaaaccatttatcatgcgggtgatacggcgctttattctgacatgaaattgttaagcgattaccatccagatgtagcatttttgccgattggagataactttacaatgggtcctgaggatgcggcaattgcagcttcatggataaatgccaaattagttgttccaatccattacgatacgttccctgttattaaacaagatcctcatgagttcgtccaatccttaaaaggaattgaagggaaagtcttacagcctggagaaggtttggatttgtaa

SEQ ID NO: 6 - protein, 227 aa, Bacillus flexus
MVHISYHGHSVVKIETQGKTILIDPFITGNGKTDLNAEDVQADYILLTHGHNDHVGDTVAIAKRTNAQVIAIAELATYLGWKGLNVHPMSIGGAYTFDFGKVKLTQAFHGSSYTEEESKQIVYTGMPAGLLLTIEGKTIYHAGDTALYSDMKLLSDYHPDVAFLPIGDNFTMGPEDAAIAASWINAKLVVPIHYDTFPVIKQDPHEFVQSLKGIEGKVLQPGEGLDL

SEQ ID NO: 7 - DNA, 684 bp, Bacillus subtilis
atgaaagtgacatatcacggacattctgtaatcacagtggaaacgaaggaccatcatattatttttgatccgtttttaacagggaattcattaacggatataaagccggaggatgtaaaagcggatgtcatcttactaacgcacggccacaatgaccatgtcggtgatactgaacaaatcgccaagcaaaataacgcgttggttatagccccaaacgagctcgctgtgtacttaggctggaaaggcttgaatgttcatccaatgcatattggcggctcccgccagttcgattttggaaaagtgaagctcacacaggcctttcatggatcagccatcacagatgaagaaaacaaaacgatcacttacacaggcatgcctgccggtattttgctgactgtagaagataaaacgattttccacgcgggcgatacggctcttttctctgatatgaaactcattggcgaattgaatcatatcgaccttgctttcctgccgattggcgataactttacgatgggacctgaagacgccaagcttgccgctgaatggctgcgcgcaaaacaggtcgtgccggtgcattacaatacattcccggtcatcgaacaagacccagaagcattcgctgacagcctgccgggcggagtcggcaaagtcatgtcggtcggtgagacgatcgagctttaa

SEQ ID NO: 8 - protein, 227 aa, Bacillus subtilis
MKVTYHGHSVITVETKDHHIIFDPFLTGNSLTDIKPEDVKADVILLTHGHNDHVGDTEQIAKQNNALVIAPNELAVYLGWKGLNVHPMHIGGSRQFDFGKVKLTQAFHGSAITDEENKTITYTGMPAGILLTVEDKTIFHAGDTALFSDMKLIGELNHIDLAFLPIGDNFTMGPEDAKLAAEWLRAKQVVPVHYNTFPVIEQDPEAFADSLPGGVGKVMSVGETIEL

SEQ ID NO: 9 - DNA, 684 bp, Bacillus cohnii
atgaaagtatcataccacggacattcagttgtcaaagtagaaacatccgattataccattcttttcgacccatttattacgggtaacagcacgacagatttgaatgcggaagatgtaaaggtagatgtcattttattaactcacggccacaatgatcatgttggggatacggtccagttagcaaagaaaaatgacgcattagttattgcgccatttgaattagctacatttttaggctggcaaggggtaaaggtccatccaatgcatattggtggagcgcatacttttgattttggaaaagtgaaattaacacaagcatttcatggatctagctatacgaacgaagaaacgaaagaaattacatatacaggaatgcctagtggaattttattaacgatagaaggaaaaacaatttaccacgcaggagacacagcgctgttttctgatatgaagctaattggagaaagaaataatattgatcttgcctttttaccgattggtgacaattttacgatgggcattgaagacgcactgcttgcggcagaatggttaggggccaaaaaagtcgtaccaatccattacaacactttcccagtcatcgaacaagacccaactcacttcactaaccaactcccagaaggtgtcggaattaccctatcacctgggcaatctattgaaatataa

SEQ ID NO: 10 - protein, 227 aa, Bacillus cohnii
MKVSYHGHSVVKVETSDYTILFDPFITGNSTTDLNAEDVKVDVILLTHGHNDHVGDTVQLAKKNDALVIAPFELATFLGWQGVKVHPMHIGGAHTFDFGKVKLTQAFHGSSYTNEETKEITYTGMPSGILLTIEGKTIYHAGDTALFSDMKLIGERNNIDLAFLPIGDNFTMGIEDALLAAEWLGAKKVVPIHYNTFPVIEQDPTHFTNQLPEGVGITLSPGQSIEI

SEQ ID NO: 11 - DNA, 687 bp, Bacillus circulans
atgaaagtaagttaccatggccattctgttgtccagattgaaacaggcgggaaaacgattttaattgatccatttattacgggaaatgaattgactgatttaaacgcagaccacgtgaaagccgatgtcattttgttaacacatggccataatgaccatgtcggcgatacagtggcaattgccaagcgtacaggggcattggttgtcgctgtagcagaattagcaacatacatgggctttcagggagttgaaaacattcatccgatgtcgattggcggagcgtaccagtttgactttggccgtgtcaagtttacacaagccttccacggctcttcattcacagaggaagacagccaaaaaattgtctatacaggcatgccaggcggcattctcttcactgctgaaggaaagaccatctaccatgcaggcgacacagctttgttcagcgacatgaagctcattggcgagcgccatcctatcgacttggcctttattccaattggcgacaactttacaatggggccagaagatgcaaaaacagcaacggaatggattgacgccagcgtaactgttccaatccattacaacacttttccgccgattaaacaggatcctcatgcttttgttaatagccttcaaagcggcaaaggacgtgtcttggaagttggcgaaacgatagtattgtaa

SEQ ID NO: 12 - protein, 228 aa, Bacillus circulans
MKVSYHGHSVVQIETGGKTILIDPFITGNELTDLNADHVKADVILLTHGHNDHVGDTVAIAKRTGALVVAVAELATYMGFQGVENIHPMSIGGAYQFDFGRVKFTQAFHGSSFTEEDSQKIVYTGMPGGILFTAEGKTIYHAGDTALFSDMKLIGERHPIDLAFIPIGDNFTMGPEDAKTATEWIDASVTVPIHYNTFPPIKQDPHAFVNSLQSGKGRVLEVGETIVL

SEQ ID NO: 13 - DNA, 687 bp, Artificial Sequence
  Bacillus licheniformis metal-dependent hydrolase nucleotide sequence codon optimised to Yarrowia lipolytica
atgaaggttgcctaccacggccattctgtcgtcactgtcgatactggagaccatcagctcatctttgatccttttattactggaaactctctcaccgatctgaagcctgaggacgttaaggccgacgtcattctcctgacccatggtcacaacgaccatgttggagacaccattgagatcgccaagcgaaacaactcgctcgtggttgctcccaacgagctggccgtgtacctgggttggaagggactgaacgtgcatcccatgcacatcggaggttctcaccagtttgacttcggaaaggtgaagctgacccaggctttccacggatctgcttacaccgaggaagattcccagaagattgtgtacaccggaatgcccgctggaatcctgctcaccgttgagggtcgaaccatcttccatgccggcgacactggactgttctctgacatgaagctgattggcgagctgaaccacattgacctcgctttcctgcccattggcgacaacttcaccatgggacctgaggatgccaagctggctgccgagtggctgcgagctaagcaggttgtccccgtgcactactcgaccttccctgtgattgagcaggaccctcacgcttttgccgactcgctgcctggtggagtcggaaaggtgctggaggttggagagtctattgagtttaagtag

SEQ ID NO: 14 - DNA, 690 bp, Artificial Sequence
  Brevibacillus borstelensis metal-dependent hydrolase nucleotide sequence optimised to Escherichia coli
atggaaatccagtatcacggtcacagctgcatccaactgaccagcgaaggtcacagcatcattatcgatccgttcatcaccggtaacccgctggcggcgaccaaactgagcgacattcgtgttcagtacatcctgctgacccacggccaccaagatcacatcctggatgcggtggagctggcgaaacagaacgacgcgaccattatcgcgacctttgaactggcgaactatctgggctggcaaggtgcgaagacaatggcgctgaacctgggtggtagcgcgagctttccgttcggtaccgttaagatgacccaggcgtttcacagcagcggcatggtgttcgaagaggataagcgtatcgtgtacctgggtatgccgggtggcttcatcgttacaatgggcggtaaaaccgtgtatcatgcgggtgataccggtctgttcggtgacatgaagctgctgggcgagcgtcacgatattgacgcggcgtttctgccgatcggtgatgttttcacaatgggtccggaagacgcgaaaaccgcggcggaatgggttggtgcgaagttcgttgtgccgatgcattacgacacctttccgccgatcaaacaggacggtaaagcgttcgttgacagcctgaaagagaagggcatcgatggtgcggcgctgaaaccgggtgaaagcacccgtctgtaa

SEQ ID NO: 15 - protein, 229 aa, Brevibacillus borstelensis
MEIQYHGHSCIQLTSEGHSIIIDPFITGNPLAATKLSDIRVQYILLTHGHQDHILDAVELAKQNDATIIATFELANYLGWQGAKTMALNLGGSASFPFGTVKMTQAFHSSGMVFEEDKRIVYLGMPGGFIVTMGGKTVYHAGDTGLFGDMKLLGERHDIDAAFLPIGDVFTMGPEDAKTAAEWVGAKFVVPMHYDTFPPIKQDGKAFVDSLKEKGIDGAALKPGESTRL

SEQ ID NO: 16 - DNA, 681 bp, Artificial Sequence
  Micrococcus lylae metal-dependent hydrolase nucleotide sequence optimised to Escherichia coli
atggaaattttctttcacggtcagagctgcattaccttcgagcacaacggcaaaaaggtgatcgttgacccgttcatcaacggcaacaaactgagcgacctggatagcgagaccgttgaagtggactacatcgtgctgacccacggtcacggcgatcatctgggtgacaccgaagcgattgcgaaacgtaccggtgcgaaagtgatcggtaccccggaactgtgcgattatctggcgggcaaaggtgttgagaacacccatccgatgaacatcggcggtaatgttgcgtttgacttcggcaaggtgaaatatgtgcaggcgttccatagcaacagcattaccgaggcgggcgaaatcatttacctgggtatgccgtgcggtctggttttcaccctgggcgaaaaggttatctaccattgcggtgataccggtctgttcagcgacatgaagctgatcgcggagcgtcatccggcggacgtgtgcttcgttccgatcggtgacaacttcacaatgggtatcgacgatgcggcgtacgcggttaacgaactgatcaagccgaccattgcggtgccgatccactacaacaccttcccgtacattgaggtggatgttaacgcgtttgcggataaggtgcacaccaaggtgaaaatcctgcagagcggtcaaagcgttagcatctaa

SEQ ID NO: 17 - protein, 226 aa, Micrococcus lylae
MEIFFHGQSCITFEHNGKKVIVDPFINGNKLSDLDSETVEVDYIVLTHGHGDHLGDTEAIAKRTGAKVIGTPELCDYLAGKGVENTHPMNIGGNVAFDFGKVKYVQAFHSNSITEAGEIIYLGMPCGLVFTLGEKVIYHCGDTGLFSDMKLIAERHPADVCFVPIGDNFTMGIDDAAYAVNELIKPTIAVPIHYNTFPYIEVDVNAFADKVHTKVKILQSGQSVSI

SEQ ID NO: 18 - DNA, 81 bp, Artificial Sequence
  oligonucleotide oPlastBug-106
acaattcctctagaaataattttgtttaactttaagaaggagatatatccatgaaagtggcatatcatggtcattcagtgg

SEQ ID NO: 19 - DNA, 82 bp, Artificial Sequence
  oligonucleotide oPlastBug-107
ttgttagcagccggatcaagctgggatttaggtgacactatagaatactcttacttaaattcgattgactcaccgacctcaa

SEQ ID NO: 20 - DNA, 78 bp, Artificial Sequence
  oligonucleotides oPlastBug-140
acaattcctctagaaataattttgtttaactttaagaaggagatatatccatggtgcacatttcttatcacggacact

SEQ ID NO: 21 - DNA, 81 bp, Artificial Sequence
  oligonucleotide oPlastBug-141
ttgttagcagccggatcaagctgggatttaggtgacactatagaatactcttacaaatccaaaccttctccaggctgtaag

SEQ ID NO: 22 - DNA, 85 bp, Artificial Sequence
  oligonucleotide oPlastBug-220
acaattcctctagaaataattttgtttaactttaagaaggagatatatccatgaaagtgacatatcacggacattctgtaatcac

SEQ ID NO: 23 - DNA, 76 bp, Artificial Sequence
  oligonucleotide oPlastBug-221
ttgttagcagccggatcaagctgggatttaggtgacactatagaatactcttaaagctcgatcgtctcaccgaccg

SEQ ID NO: 24 - DNA, 86 bp, Artificial Sequence
  oligonucleotide oPlastBug-110
acaattcctctagaaataattttgtttaactttaagaaggagatatatccatgaaagtatcttatcatggacattcagttgtgaaa

SEQ ID NO: 25 - DNA, 89 bp, Artificial Sequence
  oligonucleotide oPlastBug-111
ttgttagcagccggatcaagctgggatttaggtgacactatagaatactcctatagtgtaatactttctccagcttctaatactttccc

SEQ ID NO: 26 - protein, 226 aa, Acinetobacter sp.
MKITYYGHSCFSVFAGGRELLFDPYVTANPLAKNIDVDAIKADYIFASHAHFDHISDLLLIAGNTDAKAVGGWELYNWFNANGLKNTHPINPGGKSDFDFGTVKSFAAVHSSSFEDGSYGGVPCGYAFKTPDGNFYYSGDTALTLDMQLVSKWGKMDFAVMPIGDGLTMGVEDAIAACHMIGVKRMIGVHYDTFGFIKINHQRAVADFRDAGIELILIEIGESRDI

SEQ ID NO: 27 - protein, 264 aa, Aneurinibacillus aneurinolyticus
MEAKESVSVLAYLFAERDAGTIEWRVRVKQRMEDESMKITYHGHSCVQVSHGGKSVIIDPFLTGNSLAKVKPEDVEVQYVLLTHAHGDHLGDALTIAKRNNATVVATHELATYMSWQGANVHPMNLGGAYTFDFGTVKMTQAFHSSAIVLEDEQRIIYGGMPGGFLITMGGKTLYHLGDTGLFSDLKLIGARNRIDVAFVPIGDNFTMGPEDALQASEWVGAELTVPVHYDTFPVIKQDPGTFVSSLEGKGLKGKVLNPGEELE

SEQ ID NO: 28 - protein, 231 aa, Bacillus agri
MLEIRFHGHSSVQLTSEGHSIMIDPFISGNPLAKTTLADIQVQYILLTHGHQDHILDAVELAKANDATIVATHELATYLDWQGVKTVSMNLGGSVTLPFGKVKMTQAFHSSGVVLDDKQQIVYMGMPGGFVIELGGKTIYHAGDTGLFGDMKLIGDRHDIDLAFLPIGDVFTMGPEDAVIAAEWIAADYVVPIHYDTFPPIKQDGDAFVATLAEKDIRGKALKPGEVLRLP

SEQ ID NO: 29 - protein, 231 aa, Bacillus amyloliquefaciens
MKTMKVTYHGHSVIQVETKTHNIIFDPFLTGNSLTNLKPEEVKADVILLTHGHNDHVGDTEQIAKQNDALVIAPNELAVYLGFKGLKVHPMHIGGARQFDFGKVKLTQAFHGSAVTDEENKTITYTGMPAGILLTIDGKTIFHAGDTALFSDMKLIGELNHIDLAFLPIGDNFTMGPEDAKLAAEWLRAKQVVPVHYNTFPVIEQDPDAFAESLPGGVGKVMAVGETIELA

SEQ ID NO: 30 - protein, 226 aa, Bacillus aryabhattai
MHISYHGHSVVKIETQGKTILIDPFITGNGQTDLNADDVQADYILLTHGHNDHVGDTVAIAKRTNAQVIAIAELATYLGWKGLNVHPMSIGGAYTFEFGKVKLTQAFHGSSYTEEDNKQIVYTGMPAGLLLTIEGKTIYHAGDTALYSDMKLLSDYHPDVAFLPIGDNFTMGPEDAAVAASWINAKLVVPIHYNTFPVIKQDPHEFVQSLKGIEGKVLNPGEGLEL

SEQ ID NO: 31 - protein, 228 aa, Bacillus mycoides
MMKVSYHGHSVVKIETNGKVILIDPFLTGNPKTDLKAEDVKVDAIILSHGHGDHVGDTVALAKKNNAVVVAPFELATFLGWQGVNTHPMHIGGSHVFDFGKVKFTQAFHGSSYIDEENKTITYTGMPAGILFTAEEKTVYHAGDTALFSDMKLIGELNNIDVAFLPIGDNFTMGPEDAVLAAKWIGAKTVVPMHYNTFPVIEQDPYQFVEKLQNCTGKVLEAGESITL

SEQ ID NO: 32 - protein, 227 aa, Bacillus pumilus
MKATYHGHSVVHIETNGYHIWIDPFLNGNKHTDIKPQDVKADVILLTHGHGDHIGDTIEIAKNNDALVIAPFELATYLGWQGVKTHPLSIGGGREFEFGKVKLTQAFHGSAIIDEDAKTITYTGMPSGILFTAEGKTIYHAGDTALFSDMKLIGELNHIELAFLPIGDNFTMGPDDARIAAEWLRAKQVVPVHYSTFPPIEQDPHAFADSLHGGVGHVLNSGQSIEI

SEQ ID NO: 33 - protein, 227 aa, Bacillus vallismortis
MKVTYHGHSVITVETKGHHIIFDPFLTGNSLTDLKPEDVKADVILLTHGHNDHVGDTEQIAKQNNALVVAPNELAVYLGWKGLNVHPMHIGGSRQFDFGKVKLTQAFHGSAITDEENKTITYTGMPAGILLTVEDKTIFHAGDTALFSDMKLIGELNHIDLAFLPIGDNFTMGPEDAKLAAEWLRAKQVVPVHYNTFPVIEQDPEAFADSLPGGVGKVMAVGETIEL

SEQ ID NO: 34 - protein, 226 aa, Bacillus vietnamensis
MKISYHGHSVVKIKTNEKTILIDPFITGNELTDLKLEDEKPDVIILTHGHNDHVGDTVELAKKNDSLVIANHELATYLSWQLVKTHPMHVGGAYEFDFGKVKFTQAFHGSGLITDGNEIIYMGMPAGILLMIEGKTIFHAGDTGLFSDMKLIGERHPIDLAFLPIGDNFTMGPEDAATAASFLKAKKVVPIHYDTFPPIKQDPHKFVEMLEDSEGEVMKAGDVIEY

SEQ ID NO: 35 - protein, 231 aa, Brevibacillus brevis
MLDIRFHGHSSVQVTSKGHSIMIDPFISGSPVAATKLEDFSVQYILLTHGHLDHILDAVELAKKNDATIVATHELATYLSWQGVKTISMNLGGSIKLPFGKVKMTQAFHSSGVVLDDEKQIVYMGMPGGFVIELGGKTIYHAGDTGLFGDMKLIGERHDIDLAFLPIGDVFTMGPEDALVAAEWVQADFVVPIHYDTFPPIKQDGEAFVAELAEKDIRGKALKPGETLRLP

SEQ ID NO: 36 - protein, 227 aa, Brevibacillus thuringiensis
MKVSYHGHSVVKIETNGKVILIDPFLTGNPTTDLKAEDVKVDVILLSHGHGDHVGDTVELAKKNNAVVVAPFELATFLSWQGVNTHPMHIGGSHEFDFGKVKFTQAFHGSSYIDEENKTITYTGMPAGILFTAEEKTVYHAGDTALFSDMKLIGELNNIDIAFLPIGDNFTMGPEDAVLAAKWVQAKTVVPMHYNTFPVIEQDPYQFVEKLQNCTGKVLEAGESITL

SEQ ID NO: 37 - protein, 226 aa, Chitinophaga sp.
MELTYYGHSTFSVVAGGKHILFDPFISGNDLAKHIRIDEIKADYIFVSHGHFDHITDVASIAKRTGATVLGIWELYDHFTKQGVQNAIPLNPGGKITLDFGTAKSVPAQHSSSFPDGSYAGVAAGFVLTTPEGNFYYSGDTALTMDMSFIPKWTKLDFAVFPIGDQLTMGPEEAIEAAKLVEVNTVFGVHYDTFDIIRIDKANTIQTFKKAGVTLHLAGIGDTITI

SEQ ID NO: 38 - protein, 289 aa, Cupriavidus necator
MTRLRAVLLATVIALLGACAAPTPPAEARRPMPAGQPTAGKAEVLWLGQAATRITTPGGKVIVVDPWLTTNPKTPPGFKQLSALGKVDLILVTHAHSDHLGDAPALARLTNAPIYNGGGLGQALVSLGMVPAAQAQRFGKSGTVMPFGPTGPRITAVHAEHSSELALKNPATGKDETHYGGEPVGYIIELENGFKIWHMGDTGLFGDMRLIGEVYKPDLVLIPIGGHFTMGPQEAAIAVRDLIRPRFAIPIHYQTSPQLSGTPEAFKAALGAGAAAGVIVPQPGEKVDF

SEQ ID NO: 39 - protein, 225 aa, Flavobacterium anhuiense
MKITYYGHASLGIEVGGKYIIVDPFITGNPQAASINLSTIKADYILLTHAHPDHVLDVDAIAKNTNAVIVSNAEIASYYAKKGFNSHPMNHGGSWKFDFGKVKYVNAIHSSSFPDGSYGGNPGGFVIEGEHKNIYIAGDTALTYDMKLIPLRTKLDLAILPIGDNFTMDIEDAIIASDFVECDKVLGYHYDTFGYIEINHEEAIRKFFDKGKDLMLLPIGDSIEL

SEQ ID NO: 40 - protein, 225 aa, Flavobacterium crocinum
MKITYYGHASLAIEVGGKNIIVDPFITGNPKASGIDINVIKADYILVTHAHGDHVLDVEAIAKNTNAVIVSNAEIATYYGNKGFNSHPMNHGGSWKFDFGKVKYVNAIHSSSFPDGTYGGNPGGFVIEGEHKNIYIAGDTALTYDMKLIPLRTELDLVILPIGDNFTMDVEDAIIASDFVECDKILGYHFDTFGYIEINHEEAIRKFFDKGKDLMLLEIGESIEL

SEQ ID NO: 41 - protein, 225 aa, Flavobacterium sp.
MKITYYGHASLGIEVGGKYIIVDPFITGNPQAASINLSTIKADYILLTHAHPDHVLDVEAIAKNTNAVIVSNAEIASYYAKKGFNSHPMNHGGSWKFDFGKVKYVNAIHSSSFPEGSYGGNPGGFVIEGEHKNIYIAGDTALTYDMKLIPLRTKLDLAILPIGDNFTMDIEDAIIASDFVECDKVLGYHYDTFGYIEINHEEAIRKFFDKGKDLMLLPIGDSIEL

SEQ ID NO: 42 - protein, 225 aa, Flavobacterium succinicans
MKITFYGHACIGIEVAGKHILVDPFITGNPKASHINVNELKADYILITHAHFDHVQDVEAIAKRTDAVIVSNWEIANYFGAKELKFHPMNHGGSWTFDFGKVKYVAAVHSSAFPDGTYGGNPGGFVIEGEHKNIYISGDTALTMDMKLIPMRTKLDLAIFPIGDCFTMDIEDAIIASDFVECDKVLGCHFDTFGFIEINHEQAIKKFFDKGKDLMLLSIGESIEL

SEQ ID NO: 43 - protein, 225 aa, Flavobacterium ummariense
MKITYYGHASLGIEVNGTSIIVDPFISANELAKHIDLNSLKADYILITHAHGDHILDVETIAKNTGATIVSNAEIAGYYEAKGFKTHPMNHGGSWQFNFGKVKYVNAIHSSSFPDGTYGGQPGGFVIEADNKNVYIAGDTALTYDMKLISMRNPLDLAILPIGDNFTMDVDDAAIAAEFLEVTTVLGYHYDTFGYIKIDHEVAKQKFANKHKELILLPVGNSMEV

SEQ ID NO: 44 - protein, 225 aa, Flavobacterium xanthum
MKITFYGHASLGIAVGGKHLLVDPYISANPKAAHVDLETLKADYILLTHAHGDHILDVEAIAKRTNAVIVSNAEIAGYYAAKGFQSHPMNHGGSWNFDFGKLKFVNAIHSSSFPDGSYGGNPGGFVIEGEHKNIYIAGDTALSMDMKLIPMRTKLDLAILPIGDNFTMDVEDAIIASDFLDCDKILGYHYDTFGFIEIDHEESIRKFFNKGKDLMLLEIGSSIEL

SEQ ID NO: 45 - protein, 230 aa, Hyphomicrobium sp.
MKITWFGHSCFRIETGGSVLLIDPFLKGNPTFEKSGIAWEKAVDGVTHVAITHGHDDHIGDAAEISKRNGAPLFANYELALHVGSKGAEKLQPMNTGGTVAVEDFELTLVDALHSSSSGGVYLGNPNGIVVTAKEGKTLYHMGDTEMFGGMSLIAEFHKPDIGIVPIGDRFTMGGKRAAFACKKFFSFKTVLPCHYGTFPGMLDPDASKFVEAMSGHNVKVPKVGETVTA

SEQ ID NO: 46 - protein, 227 aa, Lysinibacillus fusiformis
MEISYHGHSVVKIQTNGKTILIDPFINGNGQTDLTVAEEAPDIIVLTHGHNDHVGDTVELAKKKDALVIAPNELANWISWQGVKAHPMHIGGAKEFDFGKVKFTQAFHGSSYVTENNEIIYMGMPAGVLLFVEGLTIYHAGDTALFSDMKLIGERHPIDIAFLPIGDNFTMGPEDAACAVSFLKPKIVVPIHYNTFPPIEQDPQIFADLVQNTEVQILKAGEKVNCF

SEQ ID NO: 47 - protein, 227 aa, Lysinibacillus mangiferihumi
MQISYHGHSVVKIQTNDQTILIDPFINGNSQTDLKVTDEAPDIILLTHGHNDHVGDTVELAKKKDALVIAPNELANWISWQGVKAHPMHIGGAKQFDFGKVKFTQAFHGSSYITENNEIIYMGMPAGVLLFIEGLTIYHAGDTALFSDMKLIGERHPIDIAFIPIGDNFTMGPEDAACAVEFLKPKIVVPIHYNTFPPIEQDPQIFANLVQNAEVQILKAGEKVNCS

SEQ ID NO: 48 - protein, 226 aa, Lysinibacillus sphaericus
MEVSYHGHSIVKIKTGDKVILIDPYITGNDLTDLVVANEKPDVILLTHGHNDHVGDTIEIAKASGALVVAPNELAVWLGWQGLETHGMNIGGACEFDFGTVKFTKAFHSSSYTTDDQQLIYMGMPAGILFMAEGKTIYHAGDTSLFGDMELIGKRHDIDIAFLPIGDNFTMGPGDAAYAVELLSPKLTVPVHYNTFPPIVQNPEDFKELVKDYEVLVMEAGDVTRI

SEQ ID NO: 49 - protein, 227 aa, Lysinibacillus xylanilyticus
MEISYHGHSVVKIQSNGKTILIDPFINGNGLTDLNVADEAPDIILLTHGHNDHVGDTVELAKKKDALVIAPNELANWISWQGVKAHPMHIGGAKEFDFGKVKFTQAFHGSSYVTENNEIIYMGMPAGILLFIEGLTIYHAGDTALFSDMKLIGERHPIDIAFLPIGDNFTMGPEDAACAVSFLKPKIVVPIHYNTFPPIEQDPQIFADLVENTEVQILKAGEKVNCS

SEQ ID NO: 50 - protein, 226 aa, Macrococcus caseolyticus; identical to SEQ ID NO: 17

SEQ ID NO: 51 - protein, 236 aa, Methylobacterium sp.
MRITWFGHSAFRLDFGESHVLIDPFFTGNPAFEGGDAARARAVEGATHILITHGHGDHVGDTLAVAAETGAKVVTNYDLCMWLGSKGLTNFDPMNTGGTTDQGGFKVTLVRADHSAGMSEAGVTVPLGLPNGVVVRAPGEPTVYHMGDTDIFGDMALIQEIYRPDVLIVPIGDRFTMGAETAALAVRKFFAPRAVIPCHYGSFPIIAASADAFVAALDGSGVQVVVPHKGTAVTIA

SEQ ID NO: 52 - protein, 226 aa, Microbacterium sp.
MNISYHGHSVVKIETNGKTILVDPFITGNELTDLKAEEVQTDVIILTHGHNDHVGDTVEIAKRNDALVIAPNELANYLGWQGLNTHPMAIGGAREFDFGTVKFTQAFHSSSYTTEDNQIIYTGMPAGFLLMAEGKTIYHAGDTGLFSDMKLIGERHPIDLAFLPIGDNFTMGPEDAALAATFLKAKEVVPVHYDTFPPIKQDPQKFVDMLEGAKGRVMTAGDTLEL

SEQ ID NO: 53 - protein, 238 aa, Nesiotobacter exalbescens
MKLTYWSHSAFTVEIKDAKILFDPFFTGNPTFPEDATVASVSEDITHLVLTHGHDDHVGDAIHILKETGAQLTANFEICMWAQSRGVENINPMNSGGCVDVGPFKVALTQAQHSSATSQAGEGYTYMGNPHGVIIMADDEPTLFHMGDTDIFGDMALINEVYAPEIGIVPIGDRFTMGAKTAAMACTRFFDFKTILPCHYRTFPILDQSADHFIEEMGADGFRIADLKPGETVDVSKS

SEQ ID NO: 54 - protein, 235 aa, Ochrobactrum intermedium
MKLTWLGHAAFRVETARAVILIDPFLNGNPGAKGIDFKEVTSGVTHIALTHGHGDHVGDTVAIAREHGATVIANADLASWLGSQGVEKVDPGNTGGTVAHDGFTITFVNALHSSAMLTENGVSQALGNPNGLVFHFEDAPTLYHMGDTDIFSDMGLINELHQPEIGIVPIGDRFTMGGAVAALACQRYFNFKTVLPCHYGSFPIIDQTADKFAEGMADHRETKVLTDPAGTVHSF

SEQ ID NO: 55 - protein, 235 aa, Ochrobactrum oryzae
MKLTWLGHAAFRIETAKAVILVDPFLNGNPGAKGIDFKEATKGVTHIALTHGHGDHVGDTVAIAREHGTTVIANADLASWLGSQGVEKLDPGNTGGTVAQDGFTVTFVNALHSSAMLTENGVSQALGNPNGLVFHFEDAPTLYHMGDTDIFSDMGLINELHQPEIGIVPIGDRFTMGGAVAALACQRYFNFKTVLPCHYASFPIIDQTADKFVAGMAEHRETKVIADPAGTVHSF

SEQ ID NO: 56 - protein, 232 aa, Paenibacillus macerans
MKITYLGHSCIHLDTGKHQIIIDPFLTGNPLAGAAADEIKADYVLLTHGHSDHISDADSISRRNHAPIVAIVELANYFAHAGLQTIGMNLGGSIQLPFGKLKWVPALHSTSVTREDGVSLYFGVAAGIVLELGDLTLYHAGDTALFSDMKLIGSRRPIDLAFLPIGDHFTMGPEDALVAAEWVNAKHVVPVHYNTFELIRQDGERFVSELQKIGIQGHFLKPTQTLNTETWN

SEQ ID NO: 57 - protein, 236 aa, Paenibacillus sp.
MLNRNISFTWLGFGGFYIKTPGNKEILIDPWIKNNPSCPETIKNIEKVDYILFTHAHRDHAEDAVWLAQTTGAMVLAGWELAFILQKKGVQNVVAINKGGTRKLGDIAVTAVHADHSSAFVDDDQIIYGGEPMGYVIKFENGYTVYHAGDTNVFGDMALIAELYEPELALLPIGDAYTMSPREAAKAVRLLNIKNVIPTHFGVFDFLTGRPSELSALLSDIEDLTVYDICPGDTIQ

SEQ ID NO: 58 - protein, 230 aa, Paracoccus yeei
MKLTWLGHSGFRLEIEQAVILIDPWMSGNPVFPDDRRDEAVRGATHILLTHGHGDHTGDTLSIARDLKIPVAGIYDLISTWQTHQGIEGIGFNKGGTVDLGGARVTMVNASHSSSFEGNEGPIYAGHESGYMIAGEGHVIYVSGDTDIMADMGWMGEYHQPDIGILSAGGHFTMDMKRAAWAARKYFDFKTVIPCHYKTFPLLEQDAQALRDGLPGVQVIEPEVLVPIAL

SEQ ID NO: 59 - protein, 234 aa, Pseudomonas sp.
MKITWLGHSAFRLDSATASILIDPFLSYNPSFAGLDLKDQTKGVTHILLTHGHGDHVGDTVQLAKETGATVLANADLAAWLGSKGLDKIEMGNTGGTIGFEGFSVTFTNALHSSAQITEDGISHALGNANGLMLHFDDEASLLHMGDTDIFSDMQLINELHKPDIGLVPIGDRFTMGGAVAALACQRFFDFKTAIPCHYGTFPIIDQTPEKFVAGMEGTRTRVLTPQPGSSVEV

SEQ ID NO: 60 - protein, 308 aa, Ralstonia sp.
MITRRLPGLSRLQSCAPRLLLASAIGLLGACAASVSNPPVPAQPAAPAAATVAAPVAAGKTEVLWLGQAAMRISTPGGKVIVVDPWLTGNPKTPAAYKQLSALGKVDLILVTHAHGDHLGDAVALAKLNNAPLWNAGGLSQQLITLGQLPATQAMRFGKGGTIAPLGDNGPKITAVHAEHSSELVWKNPATGKDETHYGGEPVGYIIEMENGFKIWHMGDTGLYGDMMMVADRYKPDLVLIPIGGHFTMGPQDAAIALRDFIKPRYAIPMHYGTSPMLRGTPAELKTALGTSATTAVIVPEPGQKVDF

SEQ ID NO: 61 - protein, 233 aa, Rhodococcus sp.
MKITYFGHSAFRIETGASVILIDPFLTGNTHFKGDIGEAAKGVTHIVLSHGHADHVGDTVGLAQQNNAKVAATYELCLWLNKKGVANIEPSNTGGTLKFDDFSVTFTQAFHSSGLLEDDGTVTYLGMPNGLVFHFNDGPTVYHMGDTDIFGDMALIEELHHPKIGLVPIGDRFTMGGAVAALACRRYFDFDTIVPIHYGTFPPLDPDANKFVEAMEGDGRRIRMPNVGEAFEV

SEQ ID NO: 62 - protein, 229 aa, Staphylococcus cohnii
MKLSFHGQSTIYFEANGKKVIVDPFITGNDLSDLDAETLEVDYIILTHGHGDHFGDTIELANRNHATVIGSAELGDYLTTTKNVENVRPMNIGGKAEFDFGTVKFVQAFHSSSLTDDNGIPVYLGMPMGLILEIEGKTIYHTGDTGLFGDMKLIAERHPVDICFIPIGDNFTMGIDDASFAINQFIKPKITVPIHYNTFDLIKQNPDDFKSAVSEGEVQILNPGDAVQL

SEQ ID NO: 63 - protein, 229 aa, Staphylococcus epidermidis
MKLSFHGQSTIYFEGNGKKVIVDPFISGNDKCDLDEQTLEVDYIILTHGHADHFGDVVELANRNHATVIGSAELQGYLSTYHGVENVHGMNIGGKAKFDFGTVKFVQAFHSSSFTHDNGVPVYLGMPMGIIVEAEGKTIYHTGDTGLFSDMKLIADRHPVDVCFVPIGDNFTMGIEDASYAINEFIKPTISVPIHYNTFPLIEQDPEQFKDAVQVGEVQILKPGESVEF

SEQ ID NO: 64 - protein, 229 aa, Staphylococcus xylosus
MKLSFHGQSTIYFEANGKKVIVDPFITGNGMSDLDASTLEVDYIILTHGHGDHFGDTIELANRNHATVIGSAELGDYLTTYHNVEHVRPMNIGGKAELDFGSVKFVQAFHSSSLTDDNGVPVYLGMPMGVIIEAEGKTIYHTGDTGLFSDMKLIADRHPVDVCFIPIGDNFTMGIDDASYAINTFIKPNITVPVHYDTFELIEQDPNAFKEAVTTSEVQILKPGDAVQF

SEQ ID NO: 65 - protein, 222 aa, Streptomyces sp.
MPALRFYGHACFGLETTTLNLLIDPWLSGNPELGAIPSDLRPPTHILVTHNHRDHVGDAIELSKTYGAPIITTPSAARHYKGEGAATAPLHLGGRLPLDGASVKCVNAIHDSPLGIGETWRVELGAPCGFVVDIDKKSVYHAGDTALFGDMALFAPVDVALLPIDGRMVMEPADAVRAAQLLQAGTVVPMHWRDQDPHAFVGLLTDAGITGHVMKNGETREL

SEQ ID NO: 66 - protein, 243 aa, Xanthobacter autotrophicus
MKITWFGHAAFRLDFADKAVLIDPFFTGNPSFNSTVEEAARGVTHILLTHGHSDHVGDTVSLVEDAADASRTLPVVANPEICAYLAAKGAGNAGQMMNTGGSLDCGGFTVTMVRADHSSGGPGSPSEYLGNPTGLIIRAPGEPTVWHMGDTDIYSDMALMCEIHRPKVVFIPIGDRFTMGPAVAALAVKRFLPGVEVVVPCHYGSFPILVQDASFFAQALADHPVKVVVPGPGGSFDANITPA